(19) United States
(12) Patent Application Publication
(10) Pub. No.: US 2025/0041293 A1
(43) Pub. Date: Feb. 6, 2025

(54) AN IGF1R INHIBITOR AND AN AKT INHIBITOR FOR USE IN THE TREATMENT OF CANCER

(71) Applicants: Genome Research Limited, London (GB); Stichting Het Nederlands Kanker Instituut-Antoni Van Leeuwenhoek Ziekenhuis, Amsterdam (NL)

(72) Inventors: Elizabeth Coker, London (GB); Amy Emery, Cambridge (GB); Mathew Garnett, London (GB); Patricia Jaaks, London (GB); Howard Lightfoot, London (GB); Jose Dianes Santos, Cambridge (GB); Daniel Vis, Amsterdam (NL); Lodewyk Wessels, Amsterdam (NL); Daniel Zerbino, Cambridge (GB)

(73) Assignees: Genome Research Limited, London (GB); Stichting Het Nederlands Kanker Instituut-Antoni Van Leeuwenhoek Ziekenhuis, Amsterdam (NL)

(21) Appl. No.: 18/836,940

(22) PCT Filed: Feb. 10, 2023
(86) PCT No.: PCT/EP2023/053386

§ 371 Date: Aug. 8, 2024

(30) Foreign Application Priority Data
Feb. 11, 2022 (GB) 2201819.6

(51) Int. Cl. A61K 31/4985; A61K 31/4375; A61P 35/00

(52) U.S. Cl. CPC A61K 31/4985; A61K 31/4375; A61P 35/00

(57) ABSTRACT

The present invention relates to a combination therapy of an IGF1R inhibitor together with an Akt inhibitor for the treatment of cancer, such as colorectal cancer, ovarian cancer, and endometrial cancer.

### FIELD OF THE INVENTION

The invention relates to combination therapies for the treatment of cancer.

### BACKGROUND

Despite advances in treatment cancer continues to have a major impact on societies, families, and individuals across the world. Cancer is among the leading causes of death worldwide. According to statistics provided by the National Cancer Institute, in 2018 there were 18.1 million new cases and 9.5 million cancer-related deaths worldwide. By 2040 the number of new cancer cases per year is expected to rise to 29.5 million and the number of cancer-related deaths to 16.4 million.

The use of single-agent targeted therapies in patients with molecularly-defined tumours is improving cancer treatment. Nonetheless, many patients still lack effective treatments and pre-existing or acquired resistance limits the clinical benefit of even our most advanced medicines. Combination therapies using the growing number of targeted anti-cancer agents have potential to overcome resistance, to enhance response to existing drugs, to reduce dose limiting single agent toxicity, and to expand the range of treatments for patients.

### SUMMARY OF THE INVENTION

The invention is directed to the use of therapeutic combinations of active ingredients for the treatment of cancer in patients, and especially to the combination of an IGF1R inhibitor with an Akt inhibitor. As described herein, the inventors have observed synergy for combinations of an IGF1R inhibitor (such as linsitinib) with an Akt inhibitor (such as MK-2206) in cancer cell lines, particularly colorectal cancer cell lines.

In a first aspect, the invention may relate to a combination of an IGF1R inhibitor together with an Akt inhibitor for use in a method of treatment of cancer in a patient, wherein the cancer is selected from colorectal cancer, ovarian cancer, and endometrial cancer.

In some cases, the invention may relate to an IGF1R inhibitor for use in a method of treatment of cancer in a patient, wherein the cancer is selected from colorectal cancer, ovarian cancer and endometrial cancer, and wherein the IGF1R inhibitor is administered to the patient in combination with an Akt inhibitor.

In some cases, the invention may relate to an Akt inhibitor for use in a method of treatment of cancer in a patient, wherein the cancer is selected from colorectal cancer, ovarian cancer and endometrial cancer, and wherein the IGF1R inhibitor is administered to the patient in combination with an IGF1R inhibitor.

In some cases, the cancer is colorectal cancer.

Suitably, the IGF1R inhibitor is selected from linsitinib, GSK1904529A, XL228, BMS-536924, BMS-754807, brigatinib, AXL-1717, and KW-2450; or is an IGF1R antibody. The IGF1R antibody may be an IGF1R monoclonal antibody (mAb). The IGF1R antibody may be selected from teprotumumab, AVE-1642, ganitumab, dalotuzumab, lonigutamab ugodotin, A-12, VRDN-002, VRDN-003, ZB-011, BIIB-022, cixutumumab, figitumumab, M-590, robatumumab, XGFR-2, XGFR-4, and istiratumab.

In some cases, the IGF1R inhibitor is selected from linsitinib, GSK1904529A, XL228, BMS-536924, BMS-754807, brigatinib, AXL-1717, and KW-2450. In some cases, the IGF1R inhibitor is selected from linsitinib, GSK1904529A, XL228, BMS-536924, BMS-754807, brigatinib, and AXL-1717. In some cases, the IGF1R inhibitor is selected from linsitinib, GSK1904529A, XL228, BMS-536924, and brigatinib. In some cases, the IGF1R inhibitor is selected from linsitinib, GSK1904529A, XL228, and BMS-536924. In some cases, the IGF1R inhibitor is selected from linsitinib and GSK1904529A. In some cases, the IGF1R inhibitor is linsitinib.

Therefore, in some cases, the invention may relate to a combination of linsitinib with an Akt inhibitor for use in a method of treatment of colorectal cancer, ovarian cancer, or endometrial cancer. In some cases, the invention may relate to a combination of AXL-1717 with an Akt inhibitor for use in a method of treatment of colorectal cancer, ovarian cancer, or endometrial cancer.

In some cases, the Akt inhibitor is an allosteric Akt inhibitor. In some cases, the Akt inhibitor is an allosteric Akt inhibitor selected from MK-2206, miransertib, and BAY1125976.

In other cases, the Akt inhibitor is an ATP-competitive Akt inhibitor. In some cases, the Akt inhibitor is an ATP-competitive Akt inhibitor selected from capivasertib, afuresertib, and ipatasertib.

Suitably, the Akt inhibitor is selected from MK-2206, capivasertib, ipatasertib, afuresertib, miransertib, uprosertib, triciribine, PTX-200, TAS-117, COTI-2, LY-2503029, MK-4440, and BAY1125976. In some cases, the Akt inhibitor is selected from MK-2206, capivasertib, ipatasertib, afuresertib, miransertib, and BAY1125976. In some cases, the Akt inhibitor is selected from MK-2206, capivasertib, miransertib, and BAY1125976. In some cases, the Akt inhibitor is selected from MK-2206, ipatasertib, and afuresertib. In some cases, the Akt inhibitor is MK-2206.

Accordingly, the combination for use according to the first aspect may be the combination of linsitinib and MK-2206.

Alternatively, the combination for use according to the first aspect may be the combination of BMS-754807 and MK-2206. Alternatively, the combination for use according to the first aspect may be the combination of GSK1904529A and MK-2206. Alternatively, the combination for use according to the first aspect may be the combination of XL228 and MK-2206. Alternatively, the combination for use according to the first aspect may be the combination of BMS-536924 and afuresertib. Alternatively, the combination is brigatinib (IGF1R inhibitor) and MK-2206 (Akt inhibitor). Alternatively, the combination is linsitinib (IGF1R inhibitor) and ipatasertib (Akt inhibitor). Alternatively, the combination is linsitinib (IGF1R inhibitor) and BAY1125976 (Akt inhibitor). Alternatively, the combination is linsitinib (IGF1R inhibitor) and capivasertib (Akt inhibitor). Alternatively, the combination is linsitinib (IGF1R inhibitor) and miransertib (Akt inhibitor). Alternatively, the combination is XL228 (IGF1R inhibitor) and ipatasertib (Akt inhibitor). Alternatively, the combination is GSK1904529A (IGF1R inhibitor) and ipatasertib (Akt inhibitor). Alternatively, the combination is GSK1904529A (IGF1R inhibitor) and afuresertib (Akt inhibitor). Alternatively, the combination is linsitinib (IGF1R inhibitor) and afuresertib (Akt inhibitor). Alternatively, the combination is BMS-536924 (IGF1R inhibitor) and MK-2206 (Akt inhibitor). Alternatively, the combination is BMS-536924 (IGF1R inhibitor) and ipatasertib (Akt inhibitor). Alternatively, the combination is BMS-536924 (IGF1R inhibitor) and miransertib (Akt inhibitor). Alternatively, the combination is BMS-536924 (IGF1R inhibitor) and capivasertib (Akt inhibitor). Alternatively, the combination is BMS-536924 (IGF1R inhibitor) and BAY1125976 (Akt inhibitor). Alternatively, the combination is GSK1904529A (IGF1R inhibitor) and miransertib (Akt inhibitor). Alternatively, the combination is GSK1904529A (IGF1R inhibitor) and capivasertib (Akt inhibitor). Alternatively, the combination is GSK1904529A (IGF1R inhibitor) and BAY1125976 (Akt inhibitor). Alternatively, the combination is AXL-1717 (IGF1R inhibitor) and MK-2206 (Akt inhibitor). Alternatively, the combination is AXL-1717 (IGF1R inhibitor) and afuresertib (Akt inhibitor). Alternatively, the combination is AXL-1717 (IGF1R inhibitor) and ipatasertib (Akt inhibitor). Alternatively, the combination is AXL-1717 (IGF1R inhibitor) and miransertib (Akt inhibitor). Alternatively, the combination is AXL-1717 (IGF1R inhibitor) and capivasertib (Akt inhibitor). Alternatively, the combination is AXL-1717 (IGF1R inhibitor) and BAY1125976 (Akt inhibitor).

In some cases, the cancer is KRAS mutant cancer.

In some cases, the cancer is ARID1A mutant and/or ARID2 mutant cancer. For example, the cancer may be ARID1A mutant cancer. The cancer may be ARID2 mutant cancer. The cancer may be ARID1A mutant and ARID2 mutant cancer.

In some cases, the cancer is ARID1A mutant and/or ARID2 mutant cancer, and is IRS4 wild-type cancer. For example, the cancer may be ARID1A mutant and IRS4 wild-type cancer. The cancer may be ARID2 mutant and IRS4 wild-type cancer. The cancer may be ARID1A mutant, ARID2 mutant, and IRS4 wild-type cancer.

In some cases, the cancer is ARID1A mutant and/or ARID2 mutant, and IRS4 wild-type colorectal cancer. In some cases, the cancer is ARID1A mutant and/or ARID2 mutant, and IRS4 wild-type colorectal cancer. and the IGF1R inhibitor is linsitinib and the Akt inhibitor is MK-2206.

In some cases, the cancer is ARID1A mutant ovarian cancer. In some cases, the cancer is ARID1A mutant ovarian cancer, and the IGF1R inhibitor is linsitinib and the Akt inhibitor is MK-2206.

In some cases, the cancer is ARID1A mutant endometrial cancer. In some cases, the cancer is ARID1A mutant endometrial cancer and the IGF1R inhibitor is linsitinib and the Akt inhibitor is MK-2206.

It will be understood that the IGF1R inhibitor and the Akt inhibitor may be administered together or separately and may be administered at the same time or at different times. For example, the compounds may be administered on different days as part of a treatment cycle or treatment regimen. Suitably but not necessarily the IGF1R inhibitor and the Akt inhibitor will be formulated separately. In preferred methods, both compounds are formulated for oral administration.

The combination therapies claimed may be used both curatively and palliatively. They may lead to better patient outcomes and/or experiences when compared to other treatment regimens and additionally or alternatively may expand the treatment options available to patients.

Suitably, the patient may be a human patient.

The invention also relates to a method of treatment of cancer in a patient in need thereof, wherein the method comprises the step of administering a combination of an effective amount of an IGF1R inhibitor together with an effective amount of an Akt inhibitor to the patient, wherein the cancer is selected from colorectal cancer, ovarian cancer, and endometrial cancer.

The invention also relates to a method of treatment of cancer in a patient in need thereof, wherein the method comprises the step of administering an effective amount of an IGF1R inhibitor to the patient in combination with an effective amount of an Akt inhibitor, wherein the cancer is selected from colorectal cancer, ovarian cancer, and endometrial cancer.

The invention also relates to a method of treatment of cancer in a patient in need thereof, wherein the method comprises the step of administering an effective amount of an Akt inhibitor to the patient in combination with an effective amount of an IGF1R inhibitor, wherein the cancer is selected from colorectal cancer, ovarian cancer, and endometrial cancer.

The invention also relates to a use of a combination of an IGF1R inhibitor together with an Akt inhibitor in the manufacture of a medicament for the treatment of cancer in a patient, wherein the cancer is selected from colorectal cancer, ovarian cancer, and endometrial cancer.

The invention also relates to a use of an IGF1R inhibitor in the manufacture of a medicament for the treatment of cancer in a patient in combination with an Akt inhibitor, wherein the cancer is selected from colorectal cancer, ovarian cancer, and endometrial cancer.

The invention also relates to a use of an Akt inhibitor in the manufacture of a medicament for the treatment of cancer in a patient in combination with an IGF1R inhibitor, wherein the cancer is selected from colorectal cancer, ovarian cancer, and endometrial cancer.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1: Correlation of individual biomarkers with synergy in response to MK-2206+Linsitinib in colorectal cancer cell lines. Log ₂ ΔIC₅₀ (A) and ΔEₘₐₓ (B) are plotted against mutation status for ARID1A. Each point is the mean response of a single cell line. Box and whisker plots show the median and interquartile range. WT: wild-type; MUT: mutant. The dotted line represents the threshold above which a cell line is defined as synergistic for the combination. See Example 2.

FIG. 2: Correlation of individual biomarkers with synergy in response to MK-2206+Linsitinib in colorectal cancer cell lines. Log ₂ ΔIC₅₀ (A) and ΔEₘₐₓ (B) are plotted against mutation status for ARID2. Each point is the mean response of a single cell line. Box and whisker plots show the median and interquartile range. WT: wild-type; MUT: mutant. The dotted line represents the threshold above which a cell line is defined as synergistic for the combination. See Example 2.

FIG. 3: Correlation of individual biomarkers with synergy in response to MK-2206+Linsitinib in colorectal cancer cell lines. Log ₂ ΔIC₅₀ (A) and ΔEₘₐₓ (B) are plotted against mutation status for IRS4. Each point is the mean response of a single cell line. Box and whisker plots show the median and interquartile range. WT: wild-type; MUT: mutant. The dotted line represents the threshold above which a cell line is defined as synergistic for the combination. See Example 2.

FIG. 4: Mutation status of ARID1A, ARID2 and IRS4 in colon cancer cell lines and correlation of biomarker combinations with sensitivity to MK-2206 (anchor)+Linsitinib (library). Colon cancer cell lines are shown ordered by Log ₂ ΔlC₅₀ with boxes underneath the plot indicating either mutant (dark grey or black) or wild-type (light grey) status for the indicated genes. Each point is the mean response of a single cell line. See Example 2.

FIG. 5: Mutation status of ARID1A, ARID2 and IRS4 in colon cancer cell lines and correlation of biomarker combinations with sensitivity to MK-2206 (anchor)+Linsitinib (library). Box plots show the combined effect of biomarkers ARID1A mutation and ARID2. The box plot labelled “ARID1A or ARID2” shows the effect of MK-2206 (anchor)+Linsitinib (library) in colon cancer cell lines that have either an ARID1A mutation or an ARID2 mutation (including colon cancer cell lines with an ARID1A mutation and an ARID2 mutation). The box plot labelled “Other” shows the effect of MK-2206 (anchor)+Linsitinib (library) in all other colon cancer cell lines. Each point is the mean response of a single cell line. A two-tailed Welch's t-test was performed. Box and whisker plots show the median and interquartile range. The dotted line represents the threshold above which a cell line is defined as synergistic for the combination. See Example 2.

FIG. 6: Mutation status of ARID1A, ARID2 and IRS4 in colon cancer cell lines and correlation of biomarker combinations with sensitivity to MK-2206 (anchor)+Linsitinib (library). Box plots show the combined effect of biomarkers ARID1A mutation, ARID2 mutation and IRS4 wild-type. The box plot labelled “ARID1A/ARID2/IRS4 wt” shows the effect of MK-2206 (anchor)+Linsitinib (library) in colon cancer cell lines that have either an ARID1A mutation or an ARID2 mutation (including colon cancer cell lines with an ARID1A mutation and an ARID2 mutation) in combination with a IRS4 wild-type status. The box plot labelled “Other” shows the effect of MK-2206 (anchor)+Linsitinib (library) in all other colon cancer cell lines. Each point is the mean response of a single cell line. Box and whisker plots show the median and interquartile range. The dotted line represents the threshold above which a cell line is defined as synergistic for the combination. See Example 2.

FIG. 7: Mutation status of ARID1A, ARID2 and IRS4 in colon cancer cell lines and correlation of biomarker combinations with sensitivity to other IGF1R and Akt inhibitor combinations. For each combination, one Akt inhibitor was combined with one IGF1R inhibitor, over a discontinuous 1,000-fold (7-point) dose range in four colorectal cancer cell lines. Viability was measured after 72 h of drug treatment using CellTiter-Glo reagent. Single-agent and combination viability measurements were fitted per cell line and multiple parameters derived including single agent values and a range of synergy scores. For all concentration combination measurements, a Bliss excess was calculated by comparing the observed combination response of cells to the Bliss independence-predicted response based on monotherapy activity. The ‘Bliss window’ was reported as the highest Bliss excess value measured across the 25 possible 3×3 submatrices, or ‘windows’, across the 7×7 dose matrix. The combinations of Akt and IGF1R inhibitors shown are: (A) BMS-536924 (IGF1R inhibitor)+Afuresertib (Akt inhibitor); (B) GSK1904529A (IGF1R inhibitor)+Afuresertib (Akt inhibitor); (C) GSK1904529A (IGF1R inhibitor)+Ipatasertib (Akt inhibitor); (D) Linsitinib (IGF1R inhibitor)+Ipatasertib (Akt inhibitor); (E) XL228 (IGF1R inhibitor)+Ipatasertib (Akt inhibitor); (F) BMS-536924 (IGF1R inhibitor)+MK-2206 (Akt inhibitor); (G) Brigatinib (IGF1R inhibitor)+MK-2206 (Akt inhibitor); (H) GSK1904529A (IGF1R inhibitor)+MK-2206 (Akt inhibitor); (I) Linsitinib (IGF1R inhibitor)+MK-2206 (Akt inhibitor); and (J) XL228 (IGF1R inhibitor)+MK-2206 (Akt inhibitor). Box plots show the combined effect of biomarkers ARID1A mutation, ARID2 mutation and IRS4 wild-type. The box plot labelled “Biomarker+ve” shows the effect of the labelled IGF1R and Akt inhibitor combination in colon cancer cell lines that have either an ARID1A mutation or an ARID2 mutation (including colon cancer cell lines with an ARID1A mutation and an ARID2 mutation) in combination with a IRS4 wild-type status. The box plot labelled “Biomarker−ve” are all other colon cancer cell lines. Each point is a replicate, with 5-8 replicates per cell line. Box and whisker plots show the median and interquartile range. See Example 3.

FIG. 8: Mutation status of ARID1A in ovarian and endometrial cancer cell lines and correlation with sensitivity to MK-2206+Linsitinib. Selected ovarian (A) or endometrial (B) cancer cell lines were treated with MK-2206+Linsitinib. Synergy metrics delta Log ₂ IC₅₀ and delta Eₘₐₓ, were generated as described in Example 5. Graphs show two independent replicates for each cell line.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

### IGF1R Inhibitor

The insulin-like growth factor 1 receptor (IGF1R or IGF-1R) is a tyrosine kinase receptor, that is, it signals by causing the addition of a phosphate molecule on particular tyrosines. IGF1R is made of two alpha subunits and two beta subunits. The IGF1R signalling pathway is implicated in several cancers as IGF1R is over-expressed in cancer cells, stimulating proliferation, enabling oncogenic transformation, and suppressing apoptosis. It is thought that IGF1R possesses anti-apoptotic properties which allow cancerous cells to resist the cytotoxic properties of chemotherapeutic drugs or radiotherapy.

Suitably, the IGF1R inhibitor is selected from linsitinib, GSK1904529A, XL228, BMS-536924, BMS-754807, brigatinib, AXL-1717, and KW-2450; or may be an IGF1R antibody. In some cases, the IGF1R inhibitor is selected from linsitinib, GSK1904529A, XL228, BMS-536924, BMS-754807, brigatinib, AXL-1717, and KW-2450. In some cases, the IGF1R inhibitor is selected from linsitinib, GSK1904529A, XL228, BMS-536924, BMS-754807, brigatinib, and AXL-1717. In some cases, the IGF1R inhibitor is selected from linsitinib, GSK1904529A, XL228, BMS-536924, and brigatinib. In some cases, the IGF1R inhibitor is selected from linsitinib, GSK1904529A, XL228, and BMS-536924. In some cases, the IGF1R inhibitor is selected from linsitinib and GSK1904529A. Preferably, the IGF1R inhibitor is linsitinib.

### Linsitinib

Linsitinib is also known as OSI-906. It is a selective inhibitor of the insulin-like growth factor 1 receptor (IGF-1R or IGF1R) with an IC₅₀ of 35 nM in cell-free assays. IGF1R is over-expressed in cancer cells, stimulating proliferation, enabling oncogenic transformation, and suppressing apoptosis. That is, linsitinib is an IGF1Ri. It has the following structure:

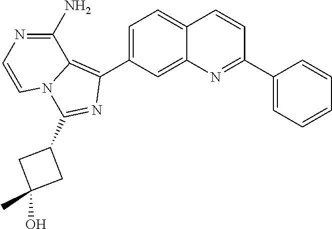 [SMILES: C[C@]1(O)C[C@@H](c2nc(-c3ccc4ccc(-c5ccccc5)nc4c3)c3c(N)nccn32)C1]

In IUPAC nomenclature it may be called 3-[8-amino-1-(2-phenyl-7-quinolinyl)-3-imidazo[1,5-a]pyrazinyl]-1-methyl-1-cyclobutanol. It is commercially available.

Linsitinib is also a potent inhibitor of the insulin receptor (IR) with an IC₅₀ of 75 nM. Without wishing to be bound by any particular theory, the inventors postulate that it is possible that the activity is mediated through dual inhibition of these targets.

### GSK1904529A

GSK1904529A is also known as GSK 4529. It is a selective inhibitor of IGF1R and IR with IC₅₀ of 27 nM and 25 nM in cell-free assays respectively, >100-fold more selective for IGF1R/IR than Akt1/2, Aurora A/B,B-Raf, CDK2, and EGFR. It has the following structure:

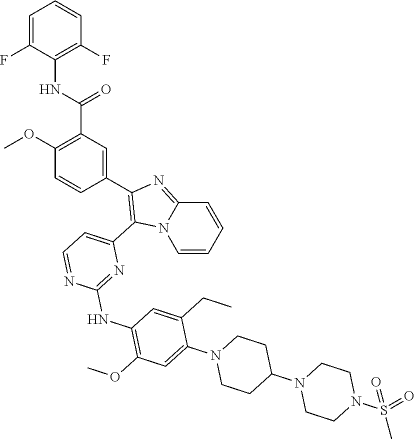 [SMILES: CCc1cc(Nc2nccc(-c3c(-c4ccc(OC)c(C(=O)Nc5c(F)cccc5F)c4)nc4ccccn34)n2)c(OC)cc1N1CCC(N2CCN(S(C)(=O)=O)CC2)CC1]

In IUPAC nomenclature it may be called N-(2,6-difluorophenyl)-5-[3-[2-[5-ethyl-2-methoxy-4-[4-(4-methylsulfonylpiperazin-1-yl)-1-piperidyl]anilino]pyrimidin-4-yl]imidazo[1,2-a]pyridine-2-yl]-2-methoxy-benzamide. It is commercially available.

### XL228

XL228 is a protein kinase inhibitor with IC₅₀ of 1.6 nM in IGF1R in cell-free assays. XL228 also exhibits an IC₅₀ of 5 nM, 1.4 nM, 3.1 nM, 6.1 nM and 2 nM for wild-type ABL kinase, ABL T3151, Aurora A, SRC and LYN in cell-free assays, respectively. It has the following structure:

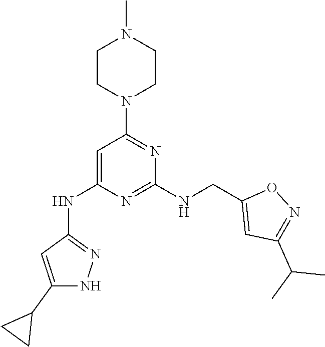 [SMILES: CC(C)c1cc(CNc2nc(Nc3cc(C4CC4)[nH]n3)cc(N3CCN(C)CC3)n2)on1]

In IUPAC nomenclature it may be called N4-(5-cyclopropyl-1H-pyrazol-3-yl)-N2-[(3-isopropylisoxazol-5-yl)methyl]-6-(4-methylpiperazin-1-yl)pyrimidine-2,4-diamine. It is commercially available.

### BMS-536924

BMS-536924 is also known as CS-0117. It is an ATP-competitive IGF1R/IR inhibitor with IC₅₀ of 100 nM/73 nM. It also has modest activity for Mek (IC₅₀ of 182 nM), Fak (IC₅₀ of 150 nM), and Lck (IC₅₀ of 341 nM) with very little activity for Akt1 and MAPK1/2. It has the following structure:

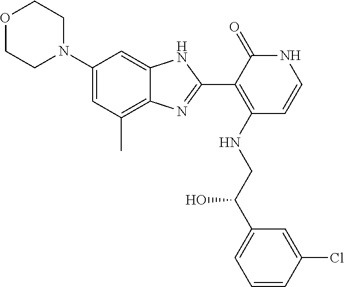 [SMILES: Cc1cc(N2CCOCC2)cc2[nH]c(-c3c(NC[C@@H](O)c4cccc(Cl)c4)cc[nH]c3=O)nc12]

In IUPAC nomenclature it may be called 4-[2-(3-chlorophenyl)-2-hydroxy-ethyl]amino]-3-(4-methyl-6-morpholino-1H-benzimidazol-2-yl)-1H-pyridin-2-one. It is commercially available.

### BMS-754807

BMS-754807 is a potent and reversible inhibitor of IGF1R/IR with IC₅₀ of 1.8 nM/1.7 nM in cell-free assays. It is less potent to Met (c-Met; IC₅₀ of 5.6 nM), Aurora A/B (IC₅₀ of 9 nM/25 nM), TrkA/B (IC₅₀ of 7.4 nM/4.1 nM) and Ron (IC₅₀ of 44 nM), and shows little activity to Flt3 (IC₅₀ of 170 nM), Lck, MK2, PKA, PKC. It has the following structure:

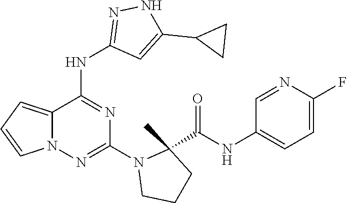 [SMILES: C[C@@]1(C(=O)Nc2ccc(F)nc2)CCCN1c1nc(Nc2cc(C3CC3)[nH]n2)c2cccn2n1]

In IUPAC nomenclature it may be called 1-[4-[(5-cyclopropyl-1H-pyrazol-3-yl)amino]pyrrolo[2,1-f][1,2,4]triazin-2-yl]-N-(6-fluoro-3-pyridyl)-2-methyl-pyrrolidine-2-carboxamide. It is commercially available.

### Brigatinib

Brigatinib is also known as AP26113. It is a potent and selective ALK (IC₅₀ of 0.6 nM) and ROS1 (IC₅₀ of 0.9 nM) inhibitor. It also inhibits IGF1R (IC₅₀ of 24.9 nM), IR (IC₅₀ of 196 nM), and FLT3 (IC₅₀ of 2.1 nM). It has the following structure:

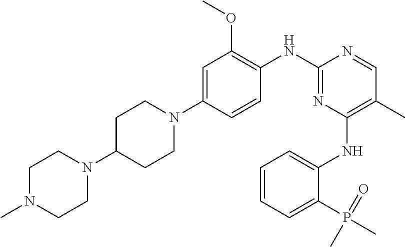

In IUPAC nomenclature it may be called 5-chloro-N4-(2-dimethylphosphorylphenyl)-N2-[2-methoxy-4-[4-(4-methylpiperazin-1-yl)-1-piperidyl]phenyl]pyrimidine-2,4-diamine. It is commercially available.

### AXL-1717

AXL-1717 is also known as AXL1717, picropodophyllin, or PPP. It is an IGF-1R inhibitor with IC₅₀ of 1 nM. It displays selectivity for IGF-1R. That is, AXL-1717 is an IGF1Ri. It does not co-inhibit tyrosine phosphorylation the IR, or of a selected panel of receptors less related to IGF-IR (for example, FGF-R, PDGF-R, or EGF-R). AXL-1717 induces apoptosis with antineoplastic activity. It has the following structure:

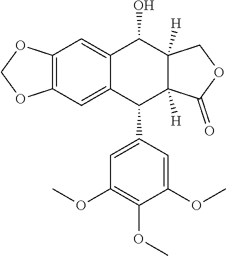 [SMILES: COc1cc([C@@H]2c3cc4c(cc3[C@H](O)[C@H]3COC(=O)[C@@H]23)OCO4)cc(OC)c1OC]

In IUPAC nomenclature it may be called (5R,5aR,8aS,9R)-5-hydroxy-9-(3,4,5-trimethoxyphenyl)-5a,6,8a,9-tetrahydro-5H-[2]benzofuro[5,6-f][1,3]benzodioxol-8-one. It is commercially available.

### KW-2450

KW-2450 is an orally active, multi-kinase inhibitor which inhibits both insulin-like growth factor receptor (IGF-1R) and insulin receptor (IR) with an IC₅₀ of 7.39 nM and 5.64 nM, respectively. It has the following structure:

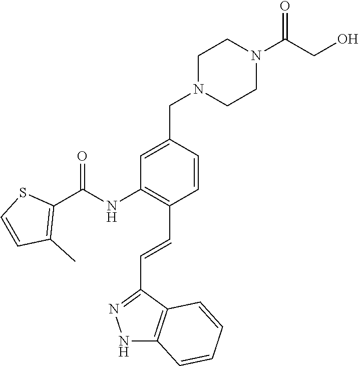 [SMILES: Cc1ccsc1C(=O)Nc1cc(CN2CCN(C(=O)CO)CC2)ccc1/C=C/c1n[nH]c2ccccc12]

In IUPAC nomenclature it may be called N-[5-[4-(2-hydroxyacetyl) piperazin-1-yl]methyl]-2-[(E)-2-(1H-indazol-3-yl) ethenyl]phenyl]-3-methylthiophene-2-carboxamide. It is commercially available.

### IGF1R Antibody

Suitably, the IGF1R inhibitor may be an IGF1R antibody. The IGF1R antibody may be an IGF1R monoclonal antibody (mAb). The IGF1R antibody may be selected from teprotumumab (Horizon Therapeutics USA Inc; Genmab AS; Horizon Therapeutics Plc; F. Hoffmann-La Roche Ltd), AVE-1642 (Viridian Therapeutics Inc; Zenas BioPharma (USA) LLC; ImmunoGen Inc), ganitumab (Amgen Inc; Takeda Pharmaceutical Co Ltd), dalotuzumab (Merck & Co Inc), lonigutamab ugodotin (Acelyrin Inc), A-12 (University of Washington), VRDN-002 (Viridian Therapeutics Inc), VRDN-003 (Viridian Therapeutics Inc), ZB-011 (Zenas BioPharma (USA) LLC), BIIB-022 (Biogen Inc), cixutumumab (Eli Lilly and Co), figitumumab (Pfizer Inc), M-590 (University of Hong Kong), robatumumab (Merck & Co Inc), XGFR-2 (F. Hoffmann-La Roche Ltd), XGFR-4 (F. Hoffmann-La Roche Ltd), and istiratumab (Merrimack Pharmaceuticals Inc).

### Akt Inhibitor

The Akt signalling pathway is implicated in inhibiting cell apoptosis and stimulating cell proliferation following the activation of Akt (also known as Protein Kinase-B)—a serine/threonine kinase. Three mammalian isoforms are currently known: Akt1/PKB-alpha, Akt2/PKB-beta, and Akt3/PKB-gamma.

Akt has been targeted both by molecules which block its ATP binding site, and by targeting so-called allosteric (other) sites (Lazaro et al. Biochem Soc Trans. 2020 Jun. 30; 48(3):933-943; Kostaras et al. Br J Cancer. 2020 August; 123 (4): 542-555). Whilst ATP-competitive Akt inhibitors (exemplified by compounds such as capivasertib, afuresertib and ipatasertib) have a predictable effect on Akt's ability to phosphorylate target proteins, simply by precluding substrate access to the active site, allosteric Akt inhibitors often have diverse and less predictable effects on enzyme function. MK-2206 (and other allosteric Akt inhibitors such as miransertib and BAY1125976) bind to Akt at a site distinct from its active site, but still exerts an inhibitory effect upon Akt kinase activity. This effect is likely due to conformational changes induced by compound binding. It is possible that such conformational changes have additional effects upon Akt activity, such as altering its ability to bind partner proteins. Allosteric Akt inhibitors may therefore exert additional effects on a target protein compared to active site blockade.

Accordingly, in some cases, the Akt inhibitor is an allosteric Akt inhibitor, such as MK-2206, miransertib, and BAY1125976.

In other cases, the Akt inhibitor is an ATP-competitive Akt inhibitor, such as capivasertib, afuresertib, and ipatasertib.

Suitably, the Akt inhibitor is selected from MK-2206, capivasertib, ipatasertib, afuresertib, miransertib, uprosertib, triciribine, PTX-200, TAS-117, COTI-2, LY-2503029, MK-4440 and BAY1125976. In some cases, the Akt inhibitor is selected from MK-2206, capivasertib, ipatasertib, afuresertib, miransertib, and BAY1125976. In some cases, the Akt inhibitor is selected from MK-2206, capivasertib, miransertib, and BAY1125976. In some cases, the Akt inhibitor is selected from MK-2206, ipatasertib, and afuresertib. Preferably, the Akt inhibitor is MK-2206.

### MK-2206

MK-2206 is also known as UNII-51HZG6MP1K. It is a pan-Akt inhibitor with IC₅₀ of 8 nM/12 nM/65 nM in cell-free assays for Akt1/2/3, respectively and has the following structure:

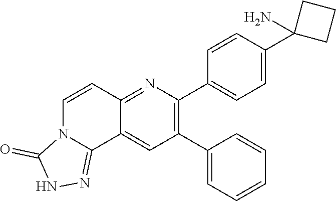 [SMILES: NC1(c2ccc(-c3nc4ccn5c(=O)[nH]nc5c4cc3-c3ccccc3)cc2)CCC1]

In IUPAC nomenclature it may be called 8-[4-(1-aminocyclobutyl)phenyl]-9-phenyl-2H-[1,2,4]triazolo[3,4-f][1,6]naphthyridin-3-one. It is commercially available, and normally supplied and used as the dihydrochloride salt.

### Capivasertib

Capivasertib, also known as AZD5363, potently inhibits all isoforms of Akt (Akt1/Akt2/Akt3) with IC₅₀ of 3 nM/8 nM/8 nM in cell-free assays for Akt1/2/3, respectively. It has the following structure:

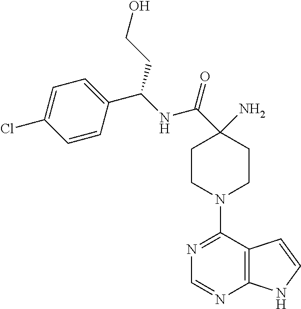 [SMILES: NC1(C(=O)N[C@@H](CCO)c2ccc(Cl)cc2)CCN(c2ncnc3[nH]ccc23)CC1]

In IUPAC nomenclature it may be called 4-amino-N-[(1S)-1-(4-chlorophenyl)-3-hydroxy-propyl]-1-(7H-pyrrolo[2,3-d]pyrimidin-4-yl) piperidine-4-carboxamide. It is commercially available.

### Ipatasertib

Ipatasertib is also known as GDC-0068 and RG7440. It is a highly selective pan-Akt inhibitor with IC₅₀ of 5 nM/18 nM/8 nM in cell-free assays for Akt1/2/3, respectively. It has the following structure:

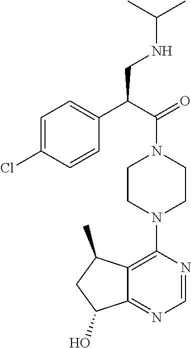 [SMILES: CC(C)NC[C@@H](C(=O)N1CCN(c2ncnc3c2[C@H](C)C[C@H]3O)CC1)c1ccc(Cl)cc1]

In IUPAC nomenclature it may be called (2S)-2-(4-chlorophenyl)-1-[4-[(5R,7R)-7-hydroxy-5-methyl-6,7-dihydro-5H-cyclopenta[d]pyrimidin-4-yl]piperazin-1-yl]-3-(isopropylamino) propan-1-one. It is commercially available.

### Afuresertib

Afuresertib is also known as GSK2110183 and ASB138. It is a potent, orally bioavailable Akt inhibitor with Kᵢ of 0.08 nM/2 nM/2.6 nM in cell-free assays for Akt1/2/3, respectively. It has the following structure:

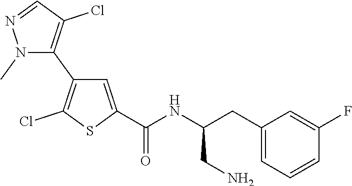 [SMILES: Cn1ncc(Cl)c1-c1cc(C(=O)N[C@H](CN)Cc2cccc(F)c2)sc1Cl]

In IUPAC nomenclature it may be called N-[(1S)-1-(aminomethyl)-2-(3-fluorophenyl)ethyl]-5-chloro-4-(4-chloro-2-methyl-pyrazol-3-yl)thiophene-2-carboxamide. It is commercially available.

### Miransertib

Miransertib, also known as ARQ-092, is a potent, selective and orally bioavailable allosteric inhibitor of Akt with IC₅₀ of 2.7 nM/14 nM/8.1 nM in cell-free assays for Akt1/2/3, respectively. It has the following structure:

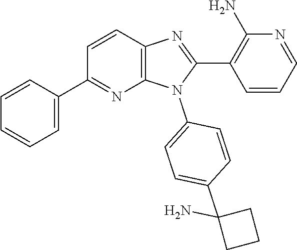 [SMILES: Nc1ncccc1-c1nc2ccc(-c3ccccc3)nc2n1-c1ccc(C2(N)CCC2)cc1]

In IUPAC nomenclature it may be called 3-[3-[4-(1-aminocyclobutyl)phenyl]-5-phenyl-imidazo[4,5-b]pyridin-2-yl]pyridin-2-amine. It is commercially available, and normally supplied and used as the hydrochloride salt.

### Uprosertib

Uprosertib is also known as GSK2141795, GSK795, and UPB795. It is a selective, ATP-competitive, and orally bioavailable Akt inhibitor with IC₅₀ of 180 nM/328 nM/38 nM in cell-free assays for Akt1/2/3, respectively. It has the following structure:

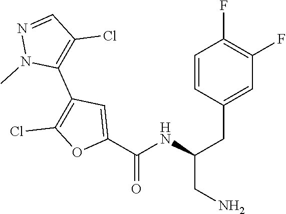 [SMILES: Cn1ncc(Cl)c1-c1cc(C(=O)N[C@H](CN)Cc2ccc(F)c(F)c2)oc1Cl]

In IUPAC nomenclature it may be called N-[(2S)-1-amino-3-(3,4-difluorophenyl) propan-2-yl]-5-chloro-4-(4-chloro-2-methylpyrazol-3-yl) furan-2-carboxamide. It is commercially available.

### Triciribine

Triciribine is also known as NSC 154020, VD-0002, vqd-002, API-2, and TCN. It is a DNA synthesis inhibitor, but also inhibits Akt in PC3 cell line (a human prostate cancer cell line) with IC₅₀ of 130 nM. It has the following structure:

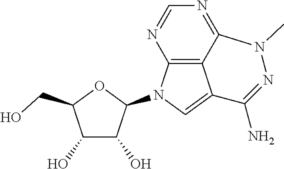 [SMILES: CN1N=C(N)c2cn([C@@H]3O[C@H](CO)[C@@H](O)[C@H]3O)c3ncnc1c23]

In IUPAC nomenclature it may be called (2R,3R,4S,5R)-2-(5-amino-7-methyl-2,6,7,9,11-pentaazatricyclo[6.3.1.0^{4,12}]dodeca-1 (12), 3,5,8,10-pentaen-2-yl)-5-(hydroxymethyl)oxolane-3,4-diol. It is commercially available, and may be supplied and used as the phosphate salt, PTX-200, described below.

### PTX-200

PTX-200 is also known as triciribine phosphate, tricycloside phosphate, and TCN-P. It is the phosphate salt of triciribine described above, which inhibits Akt in PC3 cell line (a human prostate cancer cell line) with IC₅₀ of 130 nM. It has the following structure:

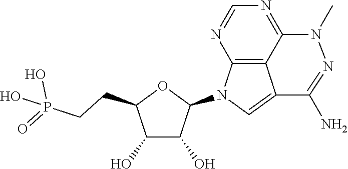 [SMILES: CN1N=C(N)c2cn([C@@H]3O[C@H](CCP(=O)(O)O)[C@@H](O)[C@H]3O)c3ncnc1c23]

In IUPAC nomenclature it may be called [(2R,3S,4R,5R)-5-(5-amino-7-methyl-2,6,7,9,11-pentaazatricyclo[6.3.1.0^{4,12}]dodeca-1 (12), 3,5,8,10-pentaen-2-yl)-3,4-dihydroxyoxolan-2-yl]methyl dihydrogen phosphate. It is commercially available, and may be supplied and used as the monohydrate, sometimes referred to as TCN-PM.

### TAS-117

TAS-117 is a potent, selective, orally active allosteric Akt inhibitor, with IC₅₀ of 4.8 nM/1.6 nM/44 nM for Akt1/2/3, respectively. TAS-117 triggers anti-myeloma activities and enhances fatal endoplasmic reticulum (ER) stress induced by proteasome inhibition. TAS-117 induces apoptosis and autophagy. It has the following structure:

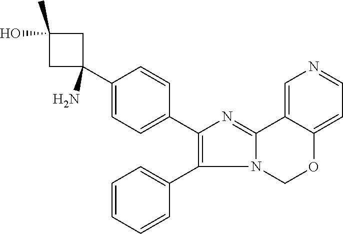 [SMILES: C[C@]1(O)C[C@@](N)(c2ccc(-c3nc4n(c3-c3ccccc3)COc3ccncc3-4)cc2)C1]

In IUPAC nomenclature it may be called 3-amino-1-methyl-3-[4-(5-phenyl-8-oxa-3,6,12-triazatricyclo[7.4.0.0^{2,6}]trideca-1(9),2,4,10,12-pentaen-4-yl)phenyl]cyclobutan-1-ol. It is commercially available, and may be supplied and used as the hydrochloride salt.

### COTI-2

COTI-2, an anti-cancer drug with low toxicity, is an orally available third generation thiosemicarbazone and activator of mutant forms of the p53 protein, with potential antineoplastic activity. COTI-2 acts both by reactivating mutant p53 and inhibiting the PI3K/Akt/mTOR pathway. COTI-2 induces apoptosis in multiple human tumour cell lines. COTI-2 exhibits antitumor activity in HNSCC through p53-dependent and -independent mechanisms. COTI-2 converts mutant p53 to wild-type conformation. It has the following structure:

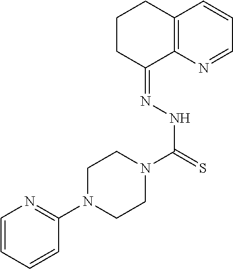 [SMILES: S=C(N/N=C1/CCCc2cccnc21)N1CCN(c2ccccn2)CC1]

In IUPAC nomenclature it may be called N—[(Z)-6,7-dihydro-5H-quinolin-8-ylideneamino]-4-pyridin-2-ylpiperazine-1-carbothioamide. It is commercially available.

### LY-2503029

LY-2503029 (Eli Lilly and Co) is protein kinase B (Akt) inhibitor. LY-2503029 binds and inhibits the activity of Akt, which result in inhibition of the PI3K/Akt signalling pathway that leads to tumour cell proliferation and the induction of tumour cell apoptosis.

### MK-4440

MK-4440 (Merck & Co Inc) is also known as MK4440, ARQ 751, and vevorisertib. It is an orally active, potent and selective pan-AKT serine/threonine kinase inhibitor, with IC₅₀ of 0.55 nM/0.81 nM/1.31 nM for Akt1/2/3, respectively. It is under development for the treatment of cancer. In particular, MK-4440, as a single agent or in combination with other anti-cancer agents, can be used for the research of solid tumours with PIK3CA/Akt/PTEN mutations. It has the following structure:

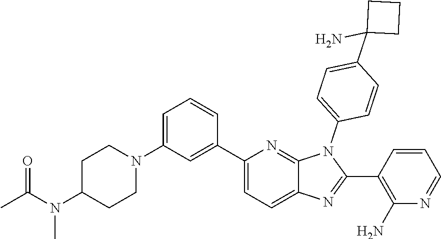 [SMILES: CC(=O)N(C)C1CCN(c2cccc(-c3ccc4nc(-c5cccnc5N)n(-c5ccc(C6(N)CCC6)cc5)c4n3)c2)CC1]

In IUPAC nomenclature it may be called N-[1-[3-[3-[4-(1-aminocyclobutyl)phenyl]-2-(2-aminopyridin-3-yl) imidazo[4,5-b]pyridin-5-yl]phenyl]piperidin-4-yl]-N-methylacetamide. It is commercially available.

### BAY1125976

BAY1125976 is a selective allosteric Akt1/Akt2 inhibitor. It inhibits Akt1 and Akt2 activity with IC₅₀ values of 5.2 nM and 18 nM at 10 UM ATP, respectively. It has the following structure:

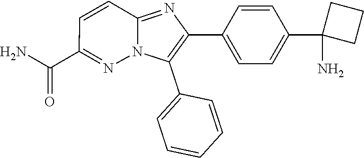

In IUPAC nomenclature it may be called 2-[4-(1-aminocyclobutyl)phenyl]-3-phenyl-imidazo[1,2-b]pyridazine-6-carboxamide. It is commercially available.

### Specific IGF1R and Akt Inhibitor Combinations

In some cases, the combination of an IGF1R inhibitor together with an Akt inhibitor for use in a method of treatment described herein may be specific IGF1R inhibitor and Akt inhibitor combinations.

In some cases, the combination may be selected from: linsitinib (IGF1R inhibitor) and MK-2206 (Akt inhibitor); BMS-754807 (IGF1R inhibitor) and MK-2206 (Akt inhibitor); GSK1904529A (IGF1R inhibitor) and MK-2206 (Akt inhibitor); XL228 (IGF1R inhibitor) and MK-2206 (Akt inhibitor); BMS-536924 (IGF1R inhibitor) and afuresertib (Akt inhibitor); BMS-536924 (IGF1R inhibitor) and MK-2206 (Akt inhibitor); brigatinib (IGF1R inhibitor) and MK-2206 (Akt inhibitor); linsitinib (IGF1R inhibitor) and ipatasertib (Akt inhibitor); linsitinib (IGF1R inhibitor) and BAY1125976 (Akt inhibitor); linsitinib (IGF1R inhibitor) and capivasertib (Akt inhibitor); linsitinib (IGF1R inhibitor) and miransertib (Akt inhibitor); XL228 (IGF1R inhibitor) and ipatasertib (Akt inhibitor); GSK1904529A (IGF1R inhibitor) and ipatasertib (Akt inhibitor); GSK1904529A (IGF1R inhibitor) and afuresertib (Akt inhibitor); linsitinib (IGF1R inhibitor) and afuresertib (Akt inhibitor); BMS-536924 (IGF1R inhibitor) and ipatasertib (Akt inhibitor); BMS-536924 (IGF1R inhibitor) and miransertib (Akt inhibitor); BMS-536924 (IGF1R inhibitor) and capivasertib (Akt inhibitor); BMS-536924 (IGF1R inhibitor) and BAY1125976 (Akt inhibitor); GSK1904529A (IGF1R inhibitor) and miransertib (Akt inhibitor); GSK1904529A (IGF1R inhibitor) and capivasertib (Akt inhibitor); GSK1904529A (IGF1R inhibitor) and BAY1125976 (Akt inhibitor); AXL-1717 (IGF1R inhibitor) and MK-2206 (Akt inhibitor); AXL-1717 (IGF1R inhibitor) and afuresertib (Akt inhibitor); AXL-1717 (IGF1R inhibitor) and ipatasertib (Akt inhibitor); AXL-1717 (IGF1R inhibitor) and miransertib (Akt inhibitor); AXL-1717 (IGF1R inhibitor) and capivasertib (Akt inhibitor); and AXL-1717 (IGF1R inhibitor) and BAY1125976 (Akt inhibitor).

In some cases, the combination may be selected from: linsitinib (IGF1R inhibitor) and MK-2206 (Akt inhibitor); BMS-754807 (IGF1R inhibitor) and MK-2206 (Akt inhibitor); GSK1904529A (IGF1R inhibitor) and MK-2206 (Akt inhibitor); XL228 (IGF1R inhibitor) and MK-2206 (Akt inhibitor); BMS-536924 (IGF1R inhibitor) and afuresertib (Akt inhibitor); BMS-536924 (IGF1R inhibitor) and MK-2206 (Akt inhibitor); brigatinib (IGF1R inhibitor) and MK-2206 (Akt inhibitor); linsitinib (IGF1R inhibitor) and ipatasertib (Akt inhibitor); linsitinib (IGF1R inhibitor) and BAY1125976 (Akt inhibitor); linsitinib (IGF1R inhibitor) and capivasertib (Akt inhibitor); linsitinib (IGF1R inhibitor) and miransertib (Akt inhibitor); XL228 (IGF1R inhibitor) and ipatasertib (Akt inhibitor); GSK1904529A (IGF1R inhibitor) and ipatasertib (Akt inhibitor); and GSK1904529A (IGF1R inhibitor) and afuresertib (Akt inhibitor).

In some cases, the combination may be selected from: linsitinib (IGF1R inhibitor) and MK-2206 (Akt inhibitor); GSK1904529A (IGF1R inhibitor) and MK-2206 (Akt inhibitor); XL228 (IGF1R inhibitor) and MK-2206 (Akt inhibitor); BMS-536924 (IGF1R inhibitor) and MK-2206 (Akt inhibitor); BMS-536924 (IGF1R inhibitor) and afuresertib (Akt inhibitor); brigatinib (IGF1R inhibitor) and MK-2206 (Akt inhibitor); linsitinib (IGF1R inhibitor) and ipatasertib (Akt inhibitor); XL228 (IGF1R inhibitor) and ipatasertib (Akt inhibitor); GSK1904529A (IGF1R inhibitor) and ipatasertib (Akt inhibitor); and GSK1904529A (IGF1R inhibitor) and afuresertib (Akt inhibitor).

In some cases, the combination may be selected from: linsitinib (IGF1R inhibitor) and MK-2206 (Akt inhibitor); BMS-754807 (IGF1R inhibitor) and MK-2206 (Akt inhibitor); and GSK1904529A (IGF1R inhibitor) and MK-2206 (Akt inhibitor).

In some cases, the combination may be selected from: linsitinib (IGF1R inhibitor) and MK-2206 (Akt inhibitor); linsitinib (IGF1R inhibitor) and BAY1125976 (Akt inhibitor); linsitinib (IGF1R inhibitor) and capivasertib (Akt inhibitor); and linsitinib (IGF1R inhibitor) and miransertib (Akt inhibitor).

In some cases, the combination is linsitinib (IGF1R inhibitor) and MK-2206 (Akt inhibitor). In some cases, the combination is BMS-754807 (IGF1R inhibitor) and MK-2206 (Akt inhibitor). In some cases, the combination is GSK1904529A (IGF1R inhibitor) and MK-2206 (Akt inhibitor). In some cases, the combination is XL228 (IGF1R inhibitor) and MK-2206 (Akt inhibitor). In some cases, the combination is BMS-536924 (IGF1R inhibitor) and afuresertib (Akt inhibitor). In some cases, the combination is BMS-536924 (IGF1R inhibitor) and MK-2206 (Akt inhibitor). In some cases, the combination is brigatinib (IGF1R inhibitor) and MK-2206 (Akt inhibitor). In some cases, the combination is linsitinib (IGF1R inhibitor) and ipatasertib (Akt inhibitor). In some cases, the combination is linsitinib (IGF1R inhibitor) and BAY1125976 (Akt inhibitor). In some cases, the combination is linsitinib (IGF1R inhibitor) and capivasertib (Akt inhibitor). In some cases, the combination is linsitinib (IGF1R inhibitor) and miransertib (Akt inhibitor). In some cases, the combination is XL228 (IGF1R inhibitor) and ipatasertib (Akt inhibitor). In some cases, the combination is GSK1904529A (IGF1R inhibitor) and ipatasertib (Akt inhibitor). In some cases, the combination is GSK1904529A (IGF1R inhibitor) and afuresertib (Akt inhibitor). In some cases, the combination is linsitinib (IGF1R inhibitor) and afuresertib (Akt inhibitor). In some cases, the combination is BMS-536924 (IGF1R inhibitor) and ipatasertib (Akt inhibitor). In some cases, the combination is BMS-536924 (IGF1R inhibitor) and miransertib (Akt inhibitor). In some cases, the combination is BMS-536924 (IGF1R inhibitor) and capivasertib (Akt inhibitor). In some cases, the combination is BMS-536924 (IGF1R inhibitor) and BAY1125976 (Akt inhibitor). In some cases, the combination is GSK1904529A (IGF1R inhibitor) and miransertib (Akt inhibitor). In some cases, the combination is GSK1904529A (IGF1R inhibitor) and capivasertib (Akt inhibitor). In some cases, the combination is GSK1904529A (IGF1R inhibitor) and BAY1125976 (Akt inhibitor). In some cases, the combination is AXL-1717 (IGF1R inhibitor) and MK-2206 (Akt inhibitor). In some cases, the combination is AXL-1717 (IGF1R inhibitor) and afuresertib (Akt inhibitor). In some cases, the combination is AXL-1717 (IGF1R inhibitor) and ipatasertib (Akt inhibitor). In some cases, the combination is AXL-1717 (IGF1R inhibitor) and miransertib (Akt inhibitor). In some cases, the combination is AXL-1717 (IGF1R inhibitor) and capivasertib (Akt inhibitor). In some cases, the combination is AXL-1717 (IGF1R inhibitor) and BAY1125976 (Akt inhibitor).

Accordingly, the combination for use in a method of treatment described herein may be the combination of linsitinib and MK-2206.

### Pharmaceutically Acceptable Salts

As described herein, any compound may be provided as a pharmaceutically acceptable salt, hydrate or solvate. Suitable pharmaceutically acceptable salts are known in the art and are described in, for example, in Berge et al., J Pharm Sci, 1977 66(1) p 1.

### Administration of Active Ingredients

Compounds used in the methods of the invention may be administered by any suitable route, including oral and intravenous routes. It will be understood that oral administration may be preferred. The compounds may be provided in pharmaceutical compositions comprising the compound and one or more pharmaceutically acceptable excipients. Formulation for oral administration may be in the form of a tablet or a capsule comprising a powder or liquid.

Administration is preferably in a “therapeutically effective amount” or an “effective amount” (used interchangeably), this being sufficient to show benefit to the individual. The actual amount administered, and rate and time-course of administration, will depend on the nature and severity of the disease being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

Suitable dosage regimens may be based on those previously used in clinical trials and/or approved regimens.

For example, linsitinib as a single agent has previously been administered at a dose of 150 mg orally to patients twice-daily during a phase I clinical trial (Fassnacht et al., Lancet Oncol., 2015 April; 16(4):426-435). The recommended dose of linsitinib for a phase II trial was 150 mg twice daily (Puzanov et al., Clin Cancer Res., 2015 Feb. 15; 21(4):701-711). Linsitinib has also been administered at a daily dose of 400-450 mg in combination with irinotecan (a TOP1 inhibitor; Davis et al., Oncologist, 2018 December; 23(12):1409-e140), treatments being administered in a cycle with linsitinib administered on days 1-3 every 7 days.

XL228 has been administered as a one-hour IV infusion once- or twice-weekly in patients with Ph+ leukemias who harbour the T3151 mutation or who are resistant to or intolerant of at least two prior BCR-ABL inhibitor therapies. Dose levels tested so far are 0.45, 0.9, 1.8, 3.6, 7.2 and 10.8 mg/kg once-weekly and 3.6 mg/kg twice-weekly (https://ir.exelixis.com/news-releases/news-release-details/exelixis-reports-positive-phase-1-data-xl228-ash-annual-meeting). XL228 has also been administered as weekly 1-hr IV infusions at the maximum tolerated dose (MTD) of 6.5 mg/kg in patients with solid tumours or multiple myeloma (Smith et al., Journal of Clinical Oncology 2010 28:15_suppl, 3105-3105).

Brigatinib has been administered at 180 mg once daily (QD) with a 7-day lead-in at 90 mg QD in a phase II study in patients with ALK-positive, advanced non-small-cell lung cancer until they experience objective disease progression per response evaluation criteria in solid tumours. Upon radiologic progression, patients who were receiving brigatinib 180 mg QD and had experienced toxicities no greater than grade 2 during treatment were able to elect, at the discretion of the investigator, to increase the brigatinib dose to 240 mg QD (Kim et al., Future Oncology 2021 17:14, 1709-1719).

AXL-1717 has been administered in patients with previously treated, locally advanced or metastatic non-small cell lung cancer in dosages of either 300 or 400 mg of AXL-1717 as twice daily (BID) treatment (58 patients) (Bergqvist et al., Acta Oncologica, 56:3, 441-447, DOI: 10.1080/0284186X.2016.1253866).

KW-2450 has been administered in combination with lapatinib (a dual tyrosine kinase inhibitor) and letrozole (an aromatase inhibitor) in a phase I study on patients with advanced/metastatic hormone receptor-positive, human epidermal growth factor receptor 2 (HER2)-positive breast cancer using the dose of KW-2450 25 mg/day plus lapatinib 1500 mg/day and letrozole 2.5 mg/day (Umehara et al., Therapeutic Advances in Medical Oncology. 2018; 10. Doi: 10.1177/1758835918786858).

MK-2206 has been administered to patients at dosages of 30 mg, 60 mg, 75 mg, and 90 mg on alternate days (Yap et al., J Clin Oncol., 2011 Dec. 10; 29(35):4688-4695). Another trial dosed patients with 200 mg of MK-2206 once a week (Xing et al., Breast Cancer Res., 2019 Jul. 5; 21(1):78). MK-2206 has also been administered orally at a dose of 135 mg once per week in combination with selumetinib (a protein kinase inhibitor; Chung et al., JAMA Oncol., 2017 Apr. 1; 3(4):516-522).

Capivasertib has been administered in a phase III, double-blind, randomised study assessing the efficacy of capivasertib and fulvestrant (a hormone therapy drug) in the treatment of patients with locally advanced (inoperable) or metastatic Hormone Receptor Positive, Human Epidermal Growth Factor Receptor 2 Negative (HR+/HER2−) breast cancer following recurrence or progression on or after aromatase inhibitor (AI) therapy. Capivasertib was dosed at 400 mg (2 oral tablets) BID on an intermittent weekly dosing schedule with dosages on days 1 to 4 in each week of a 28-day treatment cycle (https://clinicaltrials.gov/ct2/show/NCT04305496). Capivasertib has also been administered orally at 320 mg twice daily on a 4 days on/3 days off schedule, from day 2 each 21-day cycle in a phase II trial in patients with castration-resistant prostate cancer (Crabb et al., J Clin Oncol. 2021 Jan. 20; 39(3): 190-201).

Ipatasertib has been administered with paclitaxel (a chemotherapy medication) in a phase II clinical trial at 400 mg once per day on days 1-21 in 28-day cycles in patients with cancers that have a high prevalence of PI3K/Akt pathway activation, including triple-negative breast cancer (TNBC) (Kim et al., The Lancet Oncology, Volume 18, Issue 10, 2017, Pages 1360-1372; and Oliveira et al., Ann Oncol, 2019 Aug. 1; 30(8):1289-1297. Doi: 10.1093/annonc/mdz177).

Afuresertib has been administered with both carboplatin (a chemotherapy medication) and paclitaxel (another chemotherapy medication) in a phase IB dose escalation and expansion study in patients with recurrent platinum-resistant ovarian cancer, using a continuous oral dose of 50-150 mg of afuresertib per day (Blagden et al., Clin Cancer Res (2019) 25 (5): 1472-1478). Afuresertib has also been administered at 25-150 mg per day in patients with multiple myeloma in a phase I study to evaluate the maximum tolerated dose (MTD), which was observed to be 125 mg per day (Spencer et al., Blood (2014) 124 (14): 2190-2195).

Miransertib has been administered at a dose of either 200 mg QD, 5 days on/9 days off, or 150 mg QD, 5 days on/9 days off in combination with anastrozole (a hormone therapy) in a phase IB study in patients with PIK3CA and Akt1-mutant ER+ endometrial and ovarian cancer (Hyman et al., Cancer Res (2018) 78 (13_Supplement): CT035; https://doi.org/10.1158/1538-7445.AM2018-CT035). Miransertib has also been administered in a patient with Proteus syndrome at 10 mg orally daily (˜5 mg/m²/day), which escalated to 30 mg daily (˜15 mg/m²/day), and then to 50 mg daily (˜25 mg/m²/day) after 3 months of treatment (Biesecker et al., Cold Spring Harb Mol Case Study, 2020 Feb. 3; 6(1):a004549; doi: 10.1101/mcs.a004549). A phase I study in haematological malignancies also dosed miransertib at 15 mg/m² QD with subsequent maximum dose increase to 25 mg/m² (https://www.drugdiscoverytrends.com/arqule-reports-positive-phase-i-data-in-haematological-malignancies-study/).

Uprosertib has been administered at 50 mg QD in combination with trametinib (MEK1/MEK2 inhibitor) in patients with solid tumours likely to be sensitive to MEK and/or Akt inhibition in a phase I dose-escalation trial (Tolcher et al., Cancer Chemother Pharmacol, 2020 April; 85 (4): 673-683). Uprosertib has also been administered at a dose of up to 75 mg once a day orally in combination with dabrafenib (an anticancer medication, B-Raf inhibitor) and trametinib (MEK1/MEK2 inhibitor) in patients with stage IIIC-IV BRAF mutant cancer (https://clinicaltrials.gov/ct2/show/NCT01902173).

PTX-200 has been administered at 35 mg with 80 mg of paclitaxel per week in a phase 2a study in patients with locally advanced, HER2-negative breast cancer (https://smallcaps.com.au/prescient-therapeutics-encouraging-efficacy-results-leading-cancer-drug-candidate-ptx-200/). PTX-200 has also been administered intravenously over 1 hour at a dose of 25-55 mg/m² (with reduction to 15 mg/m² if needed) when used in combination with cytarabine (a chemotherapeutic agent) in patients with relapsed or refractory acute lymphoblastic leukemia (ALL), acute myeloid leukemia (AML), or chronic myeloid leukemia (CML) (https://clinicaltrials.gov/ct2/show/study/NCT02930109).

COTI-2 has been orally administered at doses of 0.5-3.5 mg/kg five days per week in combination with 60 mg/m² IV dose of cisplatin every three weeks in a phase 1b/2a trial in patients with solid tumours of ovarian, fallopian tube, primary peritoneal, endometrial, cervical, lung, pancreatic or colorectal cancer, or head and neck squamous cell carcinoma (https://www.globenewswire.com/en/news-release/2019/05/08/1819580/0/en/Cotinga-Pharmaceuticals-Releases-Early-Interim-Data-of-Phase-1b-2a-Combination-Trial-of-COTI-2-in-Solid-Tumors.html).

BAY1125976 has been administered orally at 60 mg BID in patients with hormone receptor-positive metastatic breast cancer, including nine patients harboring the AKT1^{E17K} mutation in a phase I study (Schneeweiss et al., Cancers 2019, 11(12), 1987; https://doi.org/10.3390/cancers11121987).

Suitable dosage regimens for other IGF1R inhibitors (such as GSK1904529A, BMS-536924, and BMS-754807) and Akt inhibitors (such as TAS-117, LY-2503029, and MK-4440) are also described in the art.

Accordingly, the active ingredients described herein may be administered in dosages of about 1 mg to about 1000 mg, such as about 5 mg to about 700 mg, such as about 10 mg to about 500 mg. The dosage may be dependent on the dosing schedule.

In some embodiments, each dose of the IGF1R inhibitor may be administered in a dosage of about 1 mg to about 1000 mg, such as about 50 mg to about 600 mg, such as about 100 mg to about 500 mg, such as about 150 mg to about 450 mg. In some embodiments, the IGF1R inhibitor may be administered in a dosage of about 150 mg. The IGF1R inhibitor administered at the above dosages may be selected from linsitinib, GSK1904529A, XL228, BMS-536924, BMS-754807, AXL-1717, and KW-2450; or an IGF1R antibody, as described herein.

In some embodiments, each dose of the Akt inhibitor may be administered in a dosage of about 1 mg to about 1000 mg, such as about 10 mg to about 500 mg, such as about 30 mg to about 300 mg, such as about 60 mg to about 200 mg. The Akt inhibitor administered at the above dosages may be selected from MK-2206, capivasertib, ipatasertib, afuresertib, miransertib, uprosertib, triciribine, PTX-200, TAS-117, COTI-2, LY-2503029, MK-4440, and BAY1125976 as described herein.

When administered in combination, the active ingredients described herein may be administered simultaneously or sequentially. In some embodiments of the combination therapy described herein, the IGF1R inhibitor and the Akt inhibitor are administered sequentially. In some embodiments, the active ingredients are administered in concurrent treatment cycles or regimens, and so on some days only one of the agents is administered.

Each of the active ingredients described herein may be independently administered orally or parentally.

In some embodiments, the IGR1R inhibitor may be administered orally. For example, the IGF1R inhibitor, such as linsitinib, GSK1904529A, XL228, BMS-536924, BMS-754807, brigatinib, AXL-1717, or KW-2450, may be administered orally.

In some embodiments, linsitinib may be administered orally. In some embodiments, GSK1904529A may be administered orally. In some embodiments, XL228 may be administered orally. In some embodiments, BMS-536924 may be administered orally. In some embodiments, BMS-754807 may be administered orally. In some embodiments, brigatinib may be administered orally. In some embodiments, AXL-1717 may be administered orally. In some embodiments, KW-2450 may be administered orally.

In some embodiments, the Akt inhibitor may be administered may be administered orally. For example, the Akt inhibitor, such as MK-2206, capivasertib, ipatasertib, afuresertib, miransertib, uprosertib, triciribine, PTX-200, TAS-117, COTI-2, LY-2503029, MK-4440, or BAY1125976, may be administered orally.

In some embodiments, MK-2206 may be administered may be administered orally. In some embodiments, capivasertib may be administered orally. In some embodiments, ipatasertib may be administered orally. In some embodiments, afuresertib may be administered orally. In some embodiments, miransertib may be administered orally. In some embodiments, uprosertib may be administered orally. In some embodiments, triciribine may be administered orally. In some embodiments, PTX-200 may be administered orally. In some embodiments, TAS-117 may be administered orally. In some embodiments, COTI-2 may be administered orally. In some embodiments, LY-2503029 may be administered orally. In some embodiments, MK-4440 may be administered orally. In some embodiments, BAY1125976 may be administered orally.

Each of the active ingredients described herein may be independently administered daily, such as once daily (QD), twice daily (BID), three times daily (TID), or four times daily (QID), or may be dosed less frequently, for example, every other day or on certain days within a 7 or 21 day cycle. Such treatment cycles are frequently used in chemotherapeutic treatment.

Suitably, the patient may be a human patient.

### Cancer Type

The invention relates to methods for the treatment of cancer in patients, and in particular the treatment of bowel (colorectal) cancer, ovarian cancer, and/or endometrial cancer. Accordingly, in some aspects the invention relates to the treatment of colon cancer (or colorectal cancer) in a patient. Alternatively, in some aspects the invention relates to the treatment of ovarian cancer in a patient. Alternatively, in some aspects the invention relates to the treatment of endometrial cancer in a patient.

Preferably, the cancer is colorectal cancer.

In some aspects of the invention, the cancer may be KRAS mutant cancer. In aspects of the invention wherein the cancer is colorectal cancer, the colorectal cancer may be KRAS mutant colorectal cancer. In aspects of the invention wherein the cancer is ovarian cancer, the ovarian cancer may be KRAS mutant ovarian cancer. In aspects of the invention wherein the cancer is endometrial cancer, the endometrial cancer may be KRAS mutant endometrial cancer.

In some aspects of the invention, the cancer may be ARID1A mutant and/or ARID2 mutant cancer, and optionally may be IRS4 wild-type cancer. In aspects of the invention wherein the cancer is colorectal cancer, the colorectal cancer may be ARID1A mutant and/or ARID2 mutant colorectal cancer, and optionally may be IRS4 wild-type colorectal cancer. In aspects of the invention wherein the cancer is ovarian cancer, the ovarian cancer may be ARID1A mutant and/or ARID2 mutant ovarian cancer, and optionally may be IRS4 wild-type ovarian cancer. In aspects of the invention wherein the cancer is endometrial cancer, the endometrial cancer may be ARID1A mutant and/or ARID2 mutant endometrial cancer, and optionally may be IRS4 wild-type endometrial cancer.

### KRAS Mutant

Suitably, in some aspects of the invention, the cancer is KRAS mutant cancer. In some aspects of the invention where the cancer is colorectal cancer, the colorectal cancer is KRAS mutant colorectal cancer. In some aspects of the invention wherein the cancer is ovarian cancer, the ovarian cancer is KRAS mutant ovarian cancer. In some aspects of the invention wherein the cancer is endometrial cancer, the endometrial cancer is KRAS mutant endometrial cancer.

In other words, the tumour is categorized as having KRAS mutation by genomic profiling. The KRAS (Kirsten rat sarcoma virus) gene is an oncogene. The HUGO Gene Nomenclature Committee Symbol report for KRAS can be found at: www.genenames.org which provides a link to its nucleotide sequence.

KRAS mutation is thought to be associated with about 40% of colorectal cancers and may be determined by tests that are known in the field. Most methods include the use of PCR to amplify the appropriate region of the KRAS gene, including exons 2 and 3, and then utilize different methods to distinguish wild-type from mutant sequences in key codons, such as 12, 13, and 61. The detection methods include nucleic acid sequencing, allele-specific PCR methods, single-strand conformational polymorphism analysis, melt-curve analysis, and probe hybridization. Tests for detection for KRAS mutations, such as Cobas® KRAS Mutation Test (Roche) and Therascreen KRAS RGQ PCR Kit (Qiagen), are FDA approved (https://www.fda.gov/medical-devices/in-vitro-diagnostics/list-cleared-or-approved-companion-diagnostic-devices-in-vitro-and-imaging-tools).

In other words, a KRAS mutant cancer is a cancer including cells having or harbouring an activating KRAS mutation. It will be appreciated that patients with colorectal cancer now routinely undergo KRAS mutational analysis.

As can be seen from Example 1, the inventors have observed desirable potency and efficacy for the claimed combination in colorectal cancer cells, and in particular in the KRAS mutant patient population.

### ARID1A and ARID2

The AT-rich interaction domain (ARID) family is a superfamily belonging to switch/sucrose non-fermentable (SWI/SNF) chromatin remodelling complexes, a sub-family of ATP-dependent chromatin remodelling complexes found in eukaryotes (Zhu et al., Cancer Biology & Therapy, 2022, Vol. 23, No. 1, 104-111). The ARID family consists of a series of members associated with basic processes of cellular function, including the modification of chromatin structure and the regulation of targeted gene transcription. All ARID family members contain a DNA-binding domain through which they could bind targeted DNA and participate in the process of DNA replication, gene expression and cell growth, differentiation, and development.

The AT-rich interaction domain 1A (ARID1A, sometimes called BAF250a) is a non-catalytic, DNA-binding subunit of the human SWI/SNF complex (a chromatin remodelling complex) (Tessiri et al., PeerJ, 2022, 10: e12750; Mullen et al., Cancer Treatment Reviews, 2021, 100, 102287). It is thought to play an important role in crucial cellular processes, including transcription, DNA replication, and DNA damage repair. A review by Mittal et al. (Mittal et al., 2020, Nat Rev Clin Oncol., 2020 Jul. 17(7):435-448) references papers showing that ATP-dependent chromatin remodeler SMARCA4 (also known as transcription activator BRG1) and ARID1A are recruited to sites of DNA damage and assist in homologous recombination (HR)-mediated DNA repair and non-homologous end joining (NHEJ). ARID1A is also thought to interact with DNA mismatch repair protein Msh2 (MutS homolog 2 or MSH2).

Mutation of ARID1A induces changes in the expression of multiple genes (e.g. cyclin dependent kinase inhibitor 1A (CDKN1A), mothers against decapentaplegic homolog 3 (SMAD family member 3 or SMAD3), DNA mismatch repair protein MIh1 (MutL protein homolog 1 or MLH1), and phosphoinositide-3-kinase-interacting protein 1 (PIK3IP1)) via chromatin remodelling dysfunction, which contributes to carcinogenesis and has been shown to cause transformation of cells associated with the phosphoinositide 3-kinases (PI3K)/protein kinase B (PKB or Akt) pathway (i.e. PI3K/Akt pathway) (Takeda et al., Oncology Reports, 2016, 35:607-613). Mutation of ARID1A may compromise DNA mismatch repair, leading to increased tumour mutational burden (TMB), programmed death-ligand 1 (PD-L1) expression, infiltration of cytotoxic T lymphocytes (CTL), and increased and sensitivity to checkpoint inhibitors. For example, ARID1A mutation has been linked to sensitivity to poly (ADP-ribose) polymerase (PARP) inhibitors (i.e. PARPi) and ataxia telangiectasia and Rad3-related protein (ATR) inhibitors (i.e. ATRi).

ARID1A is a frequently mutated tumour suppressor. Mutations in ARID1A have been linked to various cancers, such as ovarian clear cell carcinoma (Mittal et al., 2020, Nat Rev Clin Oncol., 2020 Jul. 17 (7): 435-448), endometriosis-associated ovarian carcinoma (Samartzis et al., Int. J. Mol. Sci., 2013, 14, 188824-18849), endometrial carcinoma (Takeda et al., Oncology Reports, 2016, 35:607-613), and cholangiocarcinoma (also known as bile duct cancer; via activation of the PI3K/Akt pathway; Tessiri et al., PeerJ, 2022, 10: e12750). In particular, ARID1A is mutated in 9% of colorectal cancers (Mullen et al., Cancer Treatment Reviews, 2021, 100, 102287), and ARID1A is mutated in more than 50% of all ovarian clear cell carcinomas and ovarian endometrial carcinomas. ARID1A mutant breast and endometrial cancer is associated with increased PI-3K and Akt signalling and sensitivity to PI-3K and Akt inhibitors (Takeda et al., Oncology Reports, 2016, 35: 607-613). Co-occurrence of ARID1A alterations with PI3K/Akt pathway activation has been reported in ovarian clear cell carcinoma, breast cancer, and gastric cancer (Huang et al., Mod Pathol, 2014 July; 27(7):983-90; Samartzis et al., Oncotarget. 2014 Jul. 30; 5(14):5295-303; Zhang et al., Oncotarget. 2016 Jul. 19; 7(29):46127-46141; De and Dey, Int J Mol Sci. 2019 Nov. 15; 20(22):5732).

ARID1A loss is linked with activation of the PI-3K/Akt/mTOR pathway (Mullen et al., Cancer Treatment Reviews, 2021, 100, 102287). ARID1A expression loss also leads to delayed mitosis and chromosomal segregation. Silencing of ARID1A in gastric, ovarian, glioma, and colon cancer cells has been shown to activate the phosphorylation of Akt and PI3K (Zeng et al., Head & Neck Oncology. 2013; 5(1):6; Xie et al., Tumour Biol. 2014 August; 35 (8): 7921-7; Takeda et al., Oncology Reports, 2016, 35:607-613; Zhang et al., Oncotarget. 2016 Jul. 19; 7(29):46127-46141), suggesting an interrelationship between ARID1A deficiency and PI3K/Akt pathway activation.

Interestingly, rhabdoid tumours and ARID1A mutant ovarian cell carcinoma are dependent on RTK signalling including platelet-derived growth factor receptors (PDGFRs), fibroblast growth factor receptors (FGFRs), and Met.

The AT-rich interaction domain 2 (ARID2, sometimes called BAF200) is a homologous subunit of the human SWI/SNF complex and also binds to DNA (Mullen et al., Cancer Treatment Reviews, 2021, 100, 102287). ARID2 is a subunit of the PBAF chromatin remodelling complex (part of the SWI/SNF complex family), which facilitates ligand-dependent transcriptional activation by nuclear receptors. Mutation studies have revealed ARID2 to be a significant tumour suppressor in many cancer subtypes. ARID2 mutations have also been linked to various cancers, and are particularly prevalent in hepatocellular carcinoma and melanoma. ARID2 mutations occur in urothelial cancers and melanoma and the patients may benefit from immune checkpoint inhibitors.

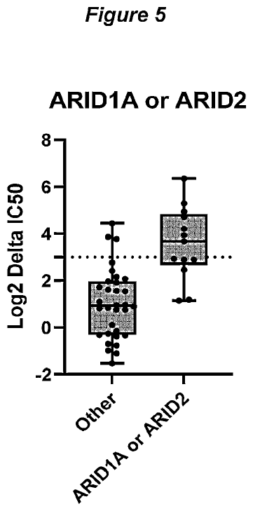

Without wishing to be bound by any particular theory, the inventors have observed that ARID1A mutant and/or ARID2 mutant colorectal cancers are particularly susceptible to treatment with combinations of an IGF1R inhibitor with an Akt inhibitor. Therefore, the inventors believe ARID1A or ARID2 mutation may provide a strong genetic biomarker for efficacy in colorectal cancer patients. See, for example, Example 2 and FIG. 5.

### IRS4

The insulin receptor substrate 4 (IRS4) gene in humans encodes the IRS4 a protein. The IRS4 protein is a cytoplasmic protein that contains many potential tyrosine and serine/threonine phosphorylation sites. The IRS4 protein is phosphorylated by the insulin receptor tyrosine kinase upon receptor stimulation.

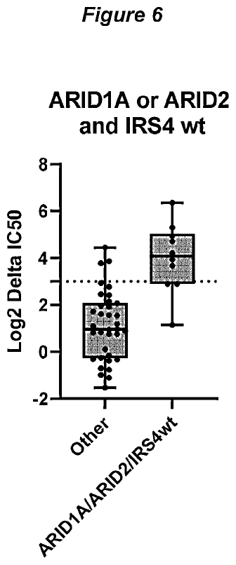

Without wishing to be bound by any particular theory, the inventors have also observed that ARID1A mutant and/or ARID2 mutant colorectal cancers which are also IRS4 wild-type colorectal cancers are particularly susceptible to treatment with combinations of an IGF1R inhibitor with an Akt inhibitor. See, for example, Example 2 and FIG. 6.

Accordingly, in some cases, the invention relates to methods for the treatment of colorectal cancer in patients, where the colorectal cancer is ARID1A mutant and/or ARID2 mutant, and IRS4 wild-type colorectal cancer.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### EXAMPLES

### Example 1

### Linsitinib or BMS-754807+MK-2206 in Breast, Colon, and Pancreas Cancer Cell Lines

Linsitinib was screened in combination with MK-2206 in 51 breast, 45 colon and 29 pancreas cancer cell lines. BMS-754807 was screened in combination with MK-2206 in 45 colon cancer cell lines. To screen efficiently we used a 2×7 concentration matrix, or “anchored” approach. We screened each anchor compound at two optimised concentrations and a discontinuous 1,000-fold (7-point) dose-response curve of the library compound. Viability was read out after 72 h of drug treatment using CellTiter-Glo and drug responses for single agent and combination responses were fitted. Single-agent and combination viability measurements were fitted per cell line and multiple parameters derived including: 1) anchor viability effect, 2) library and combination viability effect at the highest-used library concentration (library Eₘₐₓ and combo Eₘₐₓ), and 3) the estimated library drug concentration producing a 50% viability reduction (IC₅₀) for the library and combination. We compared observed combination response of cells to the Bliss independence-predicted response based on monotherapy activity, and classified drug combinations based on shifts beyond Bliss independence (Bliss, Annals of Applied Biology, 1939, 26(3), 585-615) in potency (ΔIC₅₀; i.e. increased sensitivity) or efficacy (ΔEₘₐₓ; i.e. reduced cell viability). The inventors classified combination-cell line pairs as synergistic if, at either anchor concentration, combination IC₅₀ or Eₘₐₓ was reduced 8-fold or 20% more viability reduction over Bliss, respectively. The inventors observed high rates of synergy for Linsitinib+MK-2206 in colon cancer cells (see Table 1). The combination was screened in both anchor and library orientations, the first named compound being the anchor compound.

The inventors observed that the synergy and activity was much lower with the IGF1Ri BMS-754807 (see Table 2). Synergy rate in colon was also subset to KRAS mut (n=24) and KRAS wt (n=21) cell lines (see Table 3).

TABLE 1

| Combination* | Colon | Breast | Pancreas |
|---|---|---|---|
| Linsitinib + MK-2206 | 25% | 10% | 7% |
| MK-2206 + Linsitinib | 21% | 6% | 3% |

*the first compound listed is the anchor compound. The combination was tested in both orientations.

TABLE 2

| Combination | Colon |
|---|---|
| BMS-754807 + MK-2206 | 11% |
| MK-2206 + BMS-754807 | 7% |

TABLE 3

| Combination | KRAS mut | KRAS wt |
|---|---|---|
| Linsitinib + MK-2206 | 32% | 18% |
| MK-2206 + Linsitinib | 24% | 8% |

### Example 2

### Linsitinib or BMS-754807+MK-2206 in Breast, Colon, and Pancreas Cancer Cell Lines

### Method:

Linsitinib was screened in combination with MK-2206 in 51 breast, 45 colon and 29 pancreas cancer cell lines. BMS-754807 was screened in combination with MK-2206 in 45 colon cancer cell lines. To screen efficiently we used a 2×7 concentration matrix, or “anchored” approach. The inventors screened each anchor compound at two optimised concentrations and a discontinuous 1,000-fold (7-point) dose-response curve of the library compound. Viability was read out after 72 h of drug treatment using CellTiter-Glo and drug responses for single agent and combination responses were fitted. Single-agent and combination viability measurements were fitted per cell line and multiple parameters derived including: 1) anchor viability effect, 2) library and combination viability effect at the highest-used library concentration (library Eₘₐₓ and combo Eₘₐₓ), and 3) the estimated library drug concentration producing a 50% viability reduction (IC₅₀) for the library and combination. The inventors compared observed combination response of cells to the Bliss independence-predicted response based on monotherapy activity, and classified drug combinations based on shifts beyond Bliss independence (Bliss, Annals of Applied Biology, 1939, 26(3), 585-615) in potency (ΔIC₅₀; i.e. increased sensitivity) or efficacy (ΔEₘₐₓ; i.e. reduced cell viability). The inventors classified combination-cell line pairs as synergistic if, at either anchor concentration, combination IC₅₀ or Eₘₐₓ was reduced 8-fold or 20% more viability reduction over Bliss, respectively.

### Results:

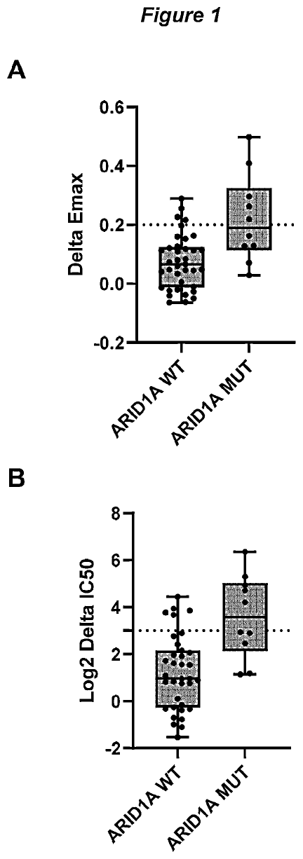

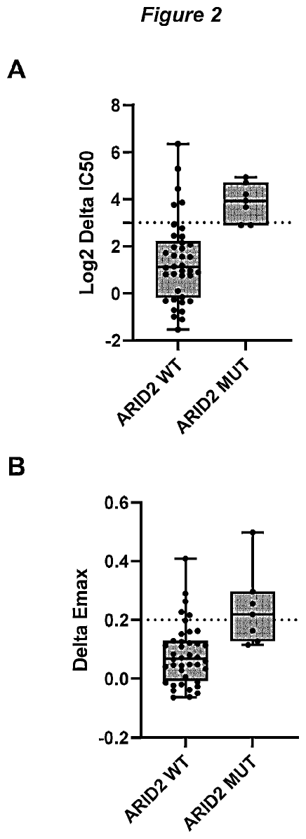

Mutation of ARID1A (n=10 for mutant; n=35 for wild-type) sensitises colon cancer cell lines to treatment with MK-2206+Linsitinib. A significantly higher shift in potency (ΔIC₅₀) and in efficacy (ΔEₘₐₓ) is observed in ARID1A mutant cell lines compared with ARID1A wild-type cells (see FIGS. 1A and 1B). This was also observed independently for ARID2 mutation (n=7 for mutant; n=38 for wild-type) (see FIGS. 2A and 2B).

The ARID1A and ARID2 mutant populations do not entirely overlap and combining the two markers as “ARID1A mutant or ARID2 mutant” (n=13) improved the ability of either biomarker alone to distinguish cell lines with high synergy from those with lower synergy (see FIG. 5).

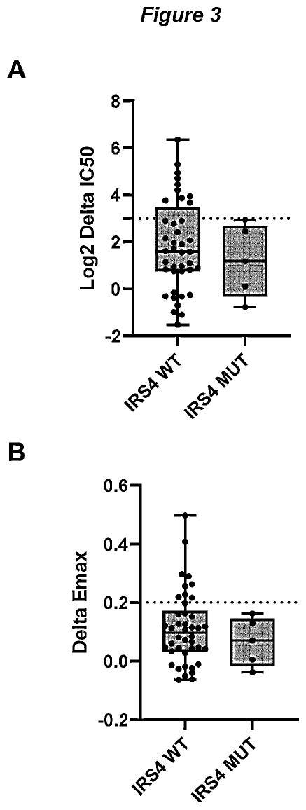

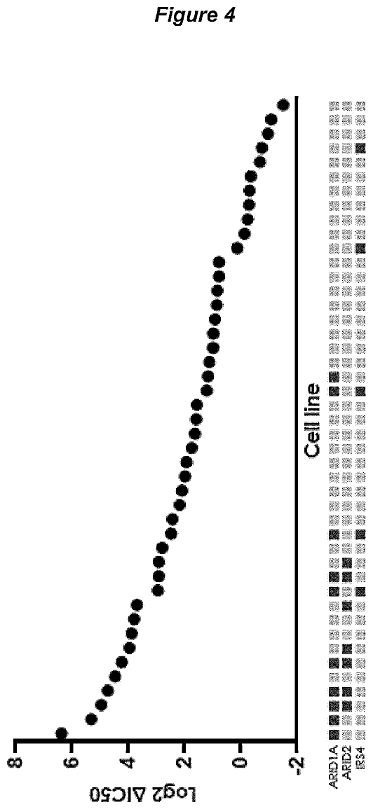

Mutation status of IRS4 alone shows some correlation with the response of colon cancer cell lines to treatment with MK-2206+Linsitinib (see FIGS. 3A and 3B). IRS4 is directly downstream of IGF1R, and IRS4 mutant cell lines are broadly less sensitive to the combination. It was observed that all IRS4 mutant cell lines had a Log ₂ ΔlC₅₀ of <3 (the cut-off for defining synergy) (see FIG. 3A and FIG. 4) raising the option to combine IRS4 with ARID1A and ARID2 as biomarkers for MK-2206+Linsitinib (see FIG. 6). Indeed, the combination of ARID1A mutant or ARID2 mutant and IRS4 wild-type improved the ability of ARID1A mutant or ARID2 mutant to distinguish cell lines with high synergy from those with lower synergy (see the comparison of FIGS. 5 and 6).

### Example 3

### Other IGF1R+Akt Inhibitor Combinations in Colon Cancer Cell Lines

### Method:

A screen was carried out in 4 colon cancer cell lines (LS-180, HCC2998, SW1417, SW837) using a 7×7 matrix approach generating 49 wells of data per cell line/drug combination. For each combination, one Akt inhibitor was combined with one IGF1R inhibitor, over a discontinuous 1,000-fold (7-point) dose range. Viability was measured after 72 h of drug treatment using CellTiter-Glo reagent. Single-agent and combination viability measurements were fitted per cell line and multiple parameters derived including single agent values and a range of synergy scores.

For all 49 concentration combination measurements a Bliss excess was calculated by comparing the observed combination response of cells to the Bliss independence-predicted response based on monotherapy activity. The “Bliss window” was reported as the highest Bliss excess value measured across the 25 possible 3×3 submatrices, or ‘windows’, across the 7×7 dose matrix.

In addition, a HSA (Highest Single Agent) excess was calculated for all 49 concentration combination measurements by comparing the observed combination response of cells to the highest single agent response for either Drug A or Drug B, whichever is highest. The “HSA window” was reported as the highest HSA excess value measured across the 25 possible 3×3 submatrices, or ‘windows’, across the 7×7 dose matrix.

### Results:

This screen included three Akt inhibitors (MK-2206, Ipatasertib and Afuresertib) each in combination with one of five IGF1R inhibitors (BMS-536924, Brigatinib, GSK1904529A, Linsitinib and XL228). For the purposes of objectively identifying synergistic combinations, cell lines were judged to show synergy to a combination if the Bliss window was greater than 0.116. This arbitrary cut-off was based on the calculation of the mean Bliss window, across all samples, plus 1× standard deviation. More generally, a higher Bliss window score indicates higher synergy.

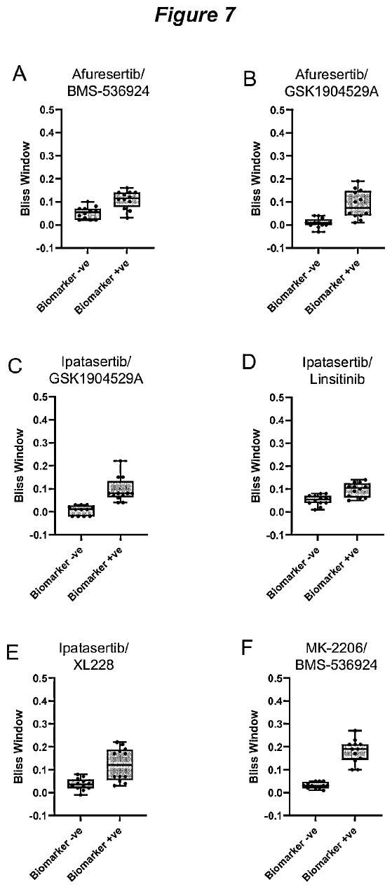

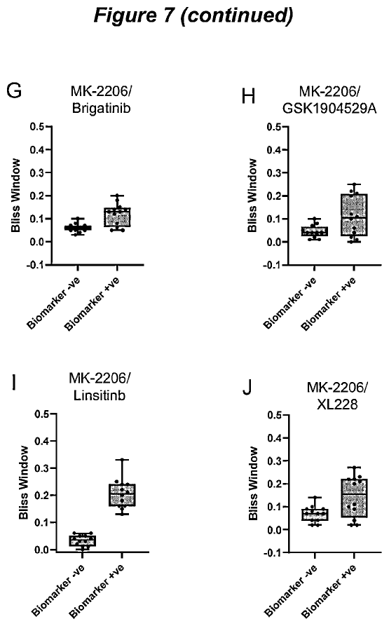

Ten combinations showed synergy in at least one of the biomarker positive cell lines (LS-180 or HCC2998) and are shown in FIG. 7(A-J). The biomarker here is ‘ARID1A mutant or ARID2 mutant and IRS4 wildtype’ as described previously. Ten combinations, shown in FIG. 7, were classified as synergistic according to the criteria above and in all cases showed increased synergy in the biomarker positive cell lines relative to biomarker negative lines.

### Example 4

### Linsitinib+Akt Inhibitor Combinations in Colon Cancer Cell Line LS-180

### Method:

Compounds were tested in the colon cancer cell line, LS-180 using a 7×7 matrix approach generating 49 wells of data per cell line/drug combination. For each combination, one Akt inhibitor was combined with one IGF1R inhibitor, over a discontinuous 1,000-fold (7-point) dose range. Viability was measured after 72 h of drug treatment using CellTiter-Glo reagent. Single-agent and combination viability measurements were fitted per cell line and multiple parameters derived including single agent values and a range of synergy scores.

Single-agent and combination viability measurements were fitted per for each combination and multiple parameters derived including: 1) single agent viability effect, 2) single agent and combination viability effect at the highest-used concentration (Eₘₐₓ), and 3) the estimated drug concentration producing a 50% viability reduction (IC₅₀) for the single agents and combination. We compared observed combination response of cells to the Bliss independence-predicted response based on monotherapy activity, and classified drug combinations based on shifts beyond Bliss independence (Bliss, Annals of Applied Biology, 1939, 26(3), 585-615) in potency (ΔIC₅₀; i.e. increased sensitivity) or efficacy (ΔEₘₐₓ; i.e. reduced cell viability).

### Results:

Four Akt inhibitors (MK-2206, Miransertib, Capivasertib, and BAY1125976) were tested in combination with a single IGF1R inhibitor (Linsitinib) in LS-180 cells. Note that this cell line is biomarker positive, where the biomarker is ARID1A mutant or ARID2 mutant and IRS4 wildtype. Table 4 below shows the maximum values achieved for ΔIC₅₀ and ΔEₘₐₓ for each combination, from two independent replicates. All four Akt inhibitors showed consistent synergy with Linsitinib in this cell line, demonstrating a minimum 5-fold change in IC₅₀.

TABLE 4

| | | Replicate 1 | | Replicate 2 | |
|---|---|---|---|---|---|
| | | Log₂ | | Log₂ | |
| Akti | IGF1Ri | ΔIC₅₀ | ΔEₘₐₓ | ΔIC₅₀ | ΔEₘₐₓ |
| MK-2206 | Linsitinib | 3.0 | 0.29 | 3.6 | 0.37 |
| BAY1125976 | Linsitinib | 2.9 | 0.31 | 2.4 | 0.32 |
| Capivasertib | Linsitinib | 3.0 | 0.29 | 2.5 | 0.24 |
| Miransertib | Linsitinib | 3.3 | 0.37 | 2.8 | 0.37 |

### Example 5

### Linsitinib and MK-2206 in Endometrial and Ovarian Cancer Cell Lines

### Method:

A screen was carried out in three endometrial cancer cell lines (MFE-280, MFE-296 and RL95-2) and three ovarian cancer cell lines (OV-90, A2780 and OAW-42) using a 7×7 matrix approach generating 49 wells of data per cell line/drug combination. MK-2206 was combined with Linsitinib, over a discontinuous 1,000-fold (7-point) dose range. Viability was measured after 72 h of drug treatment using CellTiter-Glo reagent. Single-agent and combination viability measurements were fitted per cell line and multiple parameters derived including single agent values and a range of synergy scores.

Single-agent and combination viability measurements were fitted per cell line and multiple parameters derived including: 1) anchor viability effect, 2) library and combination viability effect at the highest-used library concentration (library Eₘₐₓ and combo Eₘₐₓ), and 3) the estimated library drug concentration producing a 50% viability reduction (IC₅₀) for the library and combination. We compared observed combination response of cells to the Bliss independence-predicted response based on monotherapy activity, and classified drug combinations based on shifts beyond Bliss independence (Bliss, Annals of Applied Biology, 1939, 26(3), 585-615) in potency (ΔIC₅₀; i.e. increased sensitivity) or efficacy (ΔEₘₐₓ; i.e. reduced cell viability).

### Results:

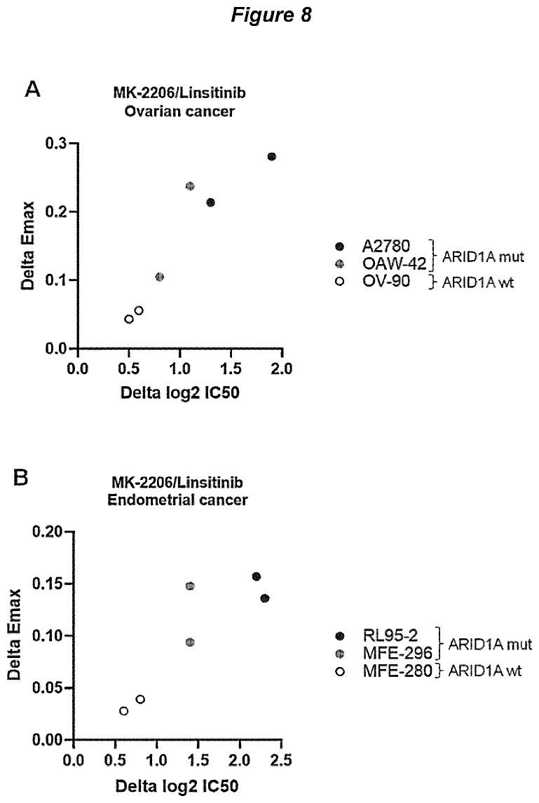

For each cancer type, two ARID1A mutant cell lines and one ARID1A wild type cell line were selected for testing. Synergy in these two cancer types was not as pronounced as for colorectal cancer, however, a correlation was seen between ARID1A mutation status and synergy. For both endometrial and ovarian cancer cell lines, Linsitinib plus MK-2206 showed greater synergy in ARID1A mutant cell lines relative to an ARID1A wild type cell line, as judged by the synergy metrics deltalC₅₀ (i.e. ΔIC₅₀) and deltaEₘₐₓ (i.e. ΔEₘₐₓ) (FIG. 8).

### REFERENCES

A number of publications are cited above in order to more fully describe and disclose the invention and the state of the art to which the invention pertains. Full citations for these references are provided below. The entirety of each of these references is incorporated herein.
Berge et al., J Pharm Sci, 1977 66(1) p 1
Bergqvist et al., Acta Oncologica, 56:3, 441-447, DOI: 10.1080/0284186X.2016.1253866
Biesecker et al., Cold Spring Harb Mol Case Study, 2020 Feb. 3; 6(1):a004549; doi: 10.1101/mcs.a004549
Blagden et al., Clin Cancer Res (2019) 25 (5): 1472-1478
Bliss, Annals of Applied Biology, 1939, 26(3), 585-615
Chung et al., JAMA Oncol., 2017 Apr. 1; 3(4):516-522
Crabb et al., J Clin Oncol. 2021 Jan. 20; 39(3): 190-201
Davis et al., Oncologist, 2018 December; 23(12):1409-e140
De and Dey, Int J Mol Sci. 2019 Nov. 15; 20(22):5732
Fassnacht et al., Lancet Oncol., 2015 April; 16 (4): 426-435
Huang et al., Mod Pathol, 2014 July; 27 (7): 983-90
Hyman et al., Cancer Res (2018) 78 (13_Supplement): CT035; https://doi.org/10.1158/1538-7445.AM2018-CT035
Kim et al., Future Oncology 2021 17:14, 1709-1719
Kim et al., The Lancet Oncology, Volume 18, Issue 10, 2017, Pages 1360-1372
Kostaras et al. Br J Cancer. 2020 August; 123 (4): 542-555
Lazaro et al. Biochem Soc Trans. 2020 Jun. 30; 48(3): 933-943
Mittal et al., 2020, Nat Rev Clin Oncol., 2020 Jul. 17(7):435-448
Mullen et al., Cancer Treatment Reviews, 2021, 100, 102287
Oliveira et al., Ann Oncol, 2019 Aug. 1; 30(8):1289-1297. doi: 10.1093/annonc/mdz177
Puzanov et al., Clin Cancer Res., 2015 Feb. 15; 21(4):701-711
Remington's Pharmaceutical Sciences, 20th Edition, 2000, pub. Lippincott, Williams & Wilkins Samartzis et al., Int. J. Mol. Sci., 2013, 14, 188824-18849
Samartzis et al., Oncotarget. 2014 Jul. 30; 5(14):5295-303
Schneeweiss et al., Cancers 2019, 11 (12), 1987; https://doi.org/10.3390/cancers11121987
Smith et al., Journal of Clinical Oncology 2010 28:15_suppl, 3105-3105
Spencer et al., Blood (2014) 124 (14): 2190-2195
Takeda et al., Oncology Reports, 2016, 35: 607-613
Tessiri et al., PeerJ, 2022, 10:e12750
Tolcher et al., Cancer Chemother Pharmacol, 2020 April; 85 (4): 673-683
Umehara et al., Therapeutic Advances in Medical Oncology. 2018; 10. doi: 10.1177/1758835918786858
Xie et al., Tumour Biol. 2014 August; 35 (8): 7921-7
Xing et al., Breast Cancer Res., 2019 Jul. 5; 21(1):78
Yap et al., J Clin Oncol., 2011 Dec. 10; 29(35):4688-4695
Zeng et al., Head & Neck Oncology. 2013; 5(1):6
Zhang et al., Oncotarget. 2016 Jul. 19; 7(29): 46127-46141
Zhu et al., Cancer Biology & Therapy, 2022, Vol. 23, No. 1, 104-111
https://clinicaltrials.gov/ct2/show/NCT01902173
https://clinicaltrials.gov/ct2/show/study/NCT02930109
https://clinicaltrials.gov/ct2/show/NCT04305496
https://www.drugdiscoverytrends.com/arqule-reports-positive-phase-i-data-in-haematological-malignancies-study/https://www.fda.gov/medical-devices/in-vitro-diagnostics/list-cleared-or-approved-companion-diagnostic-devices-in- vitro-and-imaging-tools
www.genenames.org
https://ir.exelixis.com/news-releases/news-release-details/exelixis-reports-positive-phase-1-data-x1228-ash-annual-meeting
https://www.globenewswire.com/en/news-release/2019/05/08/1819580/0/en/Cotinga-Pharmaceuticals-Releases-Early-Interim-Data-of-Phase-1b-2a-Combination-Trial-of-COTI-2-in-Solid-Tumors.html https://smallcaps.com.au/prescient-therapeutics-encouraging-efficacy-results-leading-cancer-drug-candidate-ptx-200/For standard molecular biology techniques, see Sambrook, J., Russel, D. W. Molecular Cloning, A Laboratory Manual. 3 ed. 2001, Cold Spring Harbor, New York: Cold Spring Harbor Laboratory Press.

### Priority Clauses

1. A combination of linsitinib or AXL-1717 together with an Akt inhibitor for use in a method of treatment of colorectal cancer.
2. The combination for use according to clause 1, wherein the combination is linsitinib and an Akt inhibitor.
3. The combination for use according to clause 1 or 2, wherein the Akt inhibitor is selected from MK-2206, capivasertib, ipatasertib, afuresertib, miransertib, uprosertib, triciribine, PTX-200, TAS-117, COTI-2, LY-2503029, and MK-4440.
4. The combination for use according to clause 3, wherein the Akt inhibitor is selected from MK-2206, ipatasertib, and afuresertib.
5. The combination for use according to clause 3, wherein the Akt inhibitor is MK-2206.
6. The combination for use according to clause 1, wherein the combination is linsitinib and MK-2206.
7. The combination for use according to any preceding clause, wherein the cancer is KRAS mutant colorectal cancer.
8. The combination for use according to clause 7, wherein the cancer comprises cells having an alteration at ERCC3.
9. The combination for use according to clause 8, wherein the alteration is a mutation.
10. The combination for use according to clause 8, wherein the alteration is loss of ERCC3.
11. The combination for use according to clause 10, wherein the loss of ERCC3 is homozygous loss.
12. The combination for use according to any preceding clause, wherein linsitinib or AXL-1717 and the Akt inhibitor are administered separately.

1. A combination of an IGF1R inhibitor together with an Akt inhibitor for use in a method of treatment of cancer, wherein the cancer is selected from colorectal cancer, ovarian cancer, and endometrial cancer.

2. An IGF1R inhibitor for use in a method of treatment of cancer in a patient, wherein the cancer is selected from colorectal cancer, ovarian cancer, and endometrial cancer, and wherein the IGF1R inhibitor is administered to the patient in combination with an Akt inhibitor.

3. An Akt inhibitor for use in a method of treatment of cancer in a patient, wherein the cancer is selected from colorectal cancer, ovarian cancer, and endometrial cancer, and wherein the IGF1R inhibitor is administered to the patient in combination with an IGF1R inhibitor.

4. The combination for use according to claim 1, the IGF1R inhibitor for use according to claim 2, or the Akt inhibitor for use according to claim 3, wherein the cancer is colorectal cancer.

5. The combination, the IGF1R inhibitor, or the Akt inhibitor for use according to any preceding claim, wherein the IGF1R inhibitor is selected from linsitinib, GSK1904529A, XL228, BMS-536924, BMS-754807, brigatinib, AXL-1717, and KW-2450; or is an IGF1R antibody.

6. The combination, the IGF1R inhibitor, or the Akt inhibitor for use according to claim 5, wherein the IGF1R inhibitor is selected from linsitinib, GSK1904529A, XL228, BMS-536924, BMS-754807, brigatinib, AXL-1717, and KW-2450.

7. The combination, the IGF1R inhibitor, or the Akt inhibitor for use according to claim 5, wherein the IGF1R inhibitor is selected from linsitinib, GSK1904529A, XL228, BMS-536924, BMS-754807, brigatinib, and AXL-1717.

8. The combination, the IGF1R inhibitor, or the Akt inhibitor for use according to claim 5, wherein the IGF1R inhibitor is selected from linsitinib, GSK1904529A, XL228, BMS-536924, and brigatinib.

9. The combination, the IGF1R inhibitor, or the Akt inhibitor for use according to claim 5, wherein the IGF1R inhibitor is selected from linsitinib, GSK1904529A, XL228, and BMS-536924.

10. The combination, the IGF1R inhibitor, or the Akt inhibitor for use according to claim 5, wherein the IGF1R inhibitor is selected from linsitinib and GSK1904529A.

11. The combination, the IGF1R inhibitor, or the Akt inhibitor for use according to claim 5, wherein the IGF1R inhibitor is linsitinib.

12. The combination, the IGF1R inhibitor, or the Akt inhibitor for use according to any preceding claim, wherein the Akt inhibitor is an allosteric Akt inhibitor.

13. The combination, the IGF1R inhibitor, or the Akt inhibitor for use according to any one of claims 1 to 11, wherein the Akt inhibitor is an ATP-competitive Akt inhibitor.

14. The combination, the IGF1R inhibitor, or the Akt inhibitor for use according to any one of claims 1 to 11, wherein the Akt inhibitor is selected from MK-2206, capivasertib, ipatasertib, afuresertib, miransertib, uprosertib, triciribine, PTX-200, TAS-117, COTI-2, LY-2503029, MK-4440, and BAY1125976.

15. The combination, the IGF1R inhibitor, or the Akt inhibitor for use according to claim 14, wherein the Akt inhibitor is selected from MK-2206, capivasertib, ipatasertib, afuresertib, miransertib, and BAY1125976.

16. The combination, the IGF1R inhibitor, or the Akt inhibitor for use according to claim 14, wherein the Akt inhibitor is selected from MK-2206, capivasertib, miransertib, and BAY1125976.

17. The combination, the IGF1R inhibitor, or the Akt inhibitor for use according to claim 14, wherein the Akt inhibitor is selected from MK-2206, ipatasertib, and afuresertib.

18. The combination, the IGF1R inhibitor, or the Akt inhibitor for use according to claim 14, wherein the Akt inhibitor is selected from MK-2206 and afuresertib.

19. The combination, the IGF1R inhibitor, or the Akt inhibitor for use according to claim 14, wherein the Akt inhibitor is MK-2206.

20. The combination, the IGF1R inhibitor, or the Akt inhibitor for use according to any preceding claim, wherein the IGF1R inhibitor is linsitinib and the Akt inhibitor is MK-2206.

21. The combination, the IGF1R inhibitor, or the Akt inhibitor for use according to any preceding claim, wherein the cancer is ARID1A mutant and/or ARID2 mutant cancer.

22. The combination, the IGF1R inhibitor, or the Akt inhibitor for use according to claim 21, wherein the cancer is ARID1A mutant cancer.

23. The combination, the IGF1R inhibitor, or the Akt inhibitor for use according to claim 21, wherein the cancer is ARID2 mutant cancer.

24. The combination, the IGF1R inhibitor, or the Akt inhibitor for use according to claim 21, wherein the cancer is ARID1A mutant and ARID2 mutant cancer.

25. The combination, the IGF1R inhibitor, or the Akt inhibitor for use according to any one of claims 21 to 24, wherein the cancer is IRS4 wild-type cancer.

26. The combination, the IGF1R inhibitor, or the Akt inhibitor for use according to claim 25, wherein the cancer is ARID1A mutant and/or ARID2 mutant and IRS4 wild-type colorectal cancer.

27. The combination, the IGF1R inhibitor, or the Akt inhibitor for use according to claim 22, wherein the cancer is ARID1A mutant ovarian cancer.

28. The combination, the IGF1R inhibitor, or the Akt inhibitor for use according to claim 22, wherein the cancer is ARID1A mutant endometrial cancer.

29. The combination, the IGF1R inhibitor, or the Akt inhibitor for use according to any one of claims 26 to 28, wherein the IGF1R inhibitor is linsitinib and the Akt inhibitor is MK-2206.

30. The combination, the IGF1R inhibitor, or the Akt inhibitor for use according to any preceding claim, wherein the cancer is KRAS mutant cancer.

31. The combination, the IGF1R inhibitor, or the Akt inhibitor for use according to any preceding claim, wherein the IGF1R inhibitor and the Akt inhibitor are administered separately.
